(19) United States
(12) Patent Application Publication
(10) Pub. No.: US 2024/0043915 A1
(43) Pub. Date: Feb. 8, 2024

(54) METHODS AND COMPOSITIONS FOR IN SITU MACROMOLECULE DETECTION AND USES THEREOF

(71) Applicants: THE GENERAL HOSPITAL CORPORATION, Boston, MA (US); PRESIDENT AND FELLOWS OF HARVARD COLLEGE, Cambridge, MA (US); THE BROAD INSTITUTE, INC., Cambridge, MA (US)

(72) Inventors: Evan Macosko, Boston, MA (US); Nicolas Lapique, Cambridge, MA (US); Michael Kim, Cambridge, MA (US)

(73) Assignees: THE GENERAL HOSPITAL CORPORATION, Boston, MA (US); PRESIDENT AND FELLOWS OF HARVARD COLLEGE, Cambridge, MA (US); THE BROAD INSTITUTE, INC., Cambridge, MA (US)

(21) Appl. No.: 18/546,173

(22) PCT Filed: Feb. 11, 2022
(86) PCT No.: PCT/US2022/016144

§ 371 Date: Aug. 11, 2023

Related U.S. Application Data
(60) Provisional application No. 63/149,236, filed on Feb. 13, 2021.

(51) Int. Cl. C12Q 1/6841

(52) U.S. Cl. CPC C12Q 1/6841

(57) ABSTRACT

The present disclosure relates to compositions and methods for detecting nucleic acid sequences (e.g., coding and non-coding RNAs; nuclear/genomic DNA; mtDNA; pathogen nucleic acids, etc.) in a tissue sample, specifically providing improved matrices and matrix-employing methods for performance of nucleic acid capture and amplification in a tissue sample in situ and/or in a manner that retains spatial location information for captured nucleic acids (including nucleic acid-associated macromolecules).

### CROSS-REFERENCE TO RELATED APPLICATION

The present application is related to and claims priority under 35 U.S.C. § 119(e) to U.S. provisional patent application No. 63/149,236, entitled “Methods and Compositions for In Situ Macromolecule Detection and Uses Thereof,” filed Feb. 13, 2021. The entire content of the aforementioned patent application is incorporated herein by this reference.

### STATEMENT REGARDING FEDERALLY SPONSORED RESEARCH

This invention was made with government support under Grant Nos. DP2 AG058488, 1U19MH114821, and R01HG010647, awarded by the National Institutes of Health. The government has certain rights in the invention.

### FIELD OF THE INVENTION

The invention relates generally to methods and compositions for detection of macromolecules in a tissue sample.

### BACKGROUND OF THE INVENTION

The ability to detect and identify a nucleic acid sequence of interest in a tissue sample is currently limited by the low signal capable of being achieved from endogenous, unamplified sequences. Nucleic acid amplification technologies that amplify the content of nucleic acid samples provide a solution to the limited starting materials available for analysis; however, extant in situ amplification methods, e.g., rolling circle amplification, lack control over the magnitude of production and are limited in their ability to label amplicons. Furthermore, methods that can detect proximity between biomolecules, in situ, are greatly needed to understand molecular signaling events within tissues. Therefore, a need exists for more efficient and precise approaches for nucleic acid amplification and detection in situ in tissue, as well as for improved methods of spatially robust detection of cellular macromolecules at high resolution more generally.

### BRIEF SUMMARY OF THE INVENTION

The current disclosure relates, at least in part, to discovery of compositions and methods for improved detection of macromolecules of or associated with a tissue sample, at high spatial resolution. The compositions feature monomer or polymer components in proportions capable of forming a matrix yet retaining porosity sufficient to allow for efficient enzymatic activity to occur upon matrix-attached nucleic acid primers or probes in situ. In general, such matrix components include cross-linking agents at very low concentrations as compared to other monomers or linear polymers, relative to commonly used amounts of cross-linking agents in polymeric matrices (e.g., bis-acrylamide and acrylamide, respectively, in acrylamide gel matrix formation). In certain embodiments, methods of the instant disclosure involve amplifying nucleic acid sequences (e.g., coding and non-coding RNAs; nuclear/genomic DNA; mtDNA; pathogen nucleic acids, etc., including single cell, forensic, and paleoarcheology uses, etc.) in situ in a tissue sample. Specifically contemplated applications for such improved, efficient and precise regulation of nucleic acid amplification in situ include, but are not limited to, measurement of coding and non-coding RNA sequences and amounts, detection of spatial proximity relationships between macromolecules, assessment of copy number variation (CNV), mitochondrial lineage tracing, assessment of epigenetic regulation, identification of regions of monoallelic gene expression and gene dosage in an assayed tissue, and evaluation of nucleic acid therapy deliverables to tissue, including, e.g., identification of cellular delivery of RNAi, CRISPR/Cas9 plasmid(s) and/or gels, TALEN plasmid(s) and/or gels, viral vectors (e.g., AAV), and expression vectors/plasmids, among other uses. A wide range of diagnostic, therapeutic and research applications are therefore contemplated.

In one aspect, the instant disclosure provides a composition that includes: (i) a first monomer or linear polymer; (ii) a cross-linking agent including a second monomer or polymer, where the cross-linking agent is capable of crosslinking with the first monomer or linear polymer when the cross-linking agent and the first monomer or linear polymer are combined; and (iii) a nucleic acid primer or probe having a modification capable of binding or chemically conjugating the primer or probe to the first monomer or linear polymer, to the cross-linking agent, or to both, where the ratio of the cross-linking agent to the first monomer or linear polymer is between about 1:1,000,000 and about 1:30 by weight.

In certain embodiments, the first monomer or linear polymer includes one or more of the following: acrylamide, methacrylate, polyethylene glycol (PEG), carboxymethyl cellulose (CMC), polyvinylpyrrolidone (PVP), isopropylacrylamide, hyaluronic acid, heparin, polylactic acid (PLA), polyglycolide (PGA), and poly(lactic-co-glycolic acid) (PLGA), Polyhydroxyalkanoates (PHA), propylene fumarate (PPF), agarose, alginate, chitosan, ethylene glycol-decorated polyisocyanide (PIC) polymers, derivatives thereof, and combinations thereof.

In embodiments, the cross-linking agent includes one or more of the following: N,N′-methylene bisacrylamide, trisacrylamide, tetracrylamide, polyethylene glycol dimethacrylate, amine end-functionalized 4-arm star-PEG, derivatives thereof, and combinations thereof. Optionally, the polyethylene glycol dimethacrylate includes triethylene glycol dimethyacrylate (TEGDMA), tetra(ethylene glycol) dimethacrylate, or both.

In some embodiments, the ratio of the cross-linking agent to the first monomer or linear polymer is at most 1:30 by weight. Optionally, the ratio of the cross-linking agent to the first monomer or linear polymer is at most 1:50 by weight. Optionally, the ratio of the cross-linking agent to the first monomer or linear polymer is at most 1:100 by weight. Optionally, the ratio of the cross-linking agent to the first monomer or linear polymer is at most 1:200 by weight. Optionally, the ratio of the cross-linking agent to the first monomer or linear polymer is at most 1:500 by weight. Optionally, the ratio of the cross-linking agent to the first monomer or linear polymer is at most 1:1000 by weight. Optionally, the ratio of the cross-linking agent to the first monomer or linear polymer is at most 1:2,000 by weight. Optionally, the ratio of the cross-linking agent to the first monomer or linear polymer is at most 1:3,000 by weight. Optionally, the ratio of the cross-linking agent to the first monomer or linear polymer is at most 1:5,000 by weight. Optionally, the ratio of the cross-linking agent to the first monomer or linear polymer is at most 1:10,000 by weight. Optionally, the ratio of the cross-linking agent to the first monomer or linear polymer is at most 1:30,000 by weight. Optionally, the ratio of the cross-linking agent to the first monomer or linear polymer is at most 1:50,000 by weight. Optionally, the ratio of the cross-linking agent to the first monomer or linear polymer is at most 1:100,000 by weight. Optionally, the ratio of the cross-linking agent to the first monomer or linear polymer is at most 1:300,000 by weight. Optionally, the ratio of the cross-linking agent to the first monomer or linear polymer is at most 1:500,000 by weight. Optionally, the ratio of the cross-linking agent to the first monomer or linear polymer is at most 1:750,000 by weight. Optionally, the ratio of the cross-linking reagent to the to the first monomer or linear polymer is at most 1:1,000,000 by weight.

In certain embodiments, the modification of the nucleic acid primer or probe is a phosphoramidite modification. Optionally, the modification is an acrydite modification.

In some embodiments, the nucleic acid primer or probe binds or chemically conjugates to the first monomer or linear polymer. Optionally, the nucleic acid primer or probe covalently binds or chemically conjugates to the first monomer or linear polymer. Optionally, the first monomer or linear polymer is acrylamide.

In embodiments, the composition further includes a cell or tissue. Optionally, the cell or tissue is a fixed and/or permeabilized cell or tissue.

In certain embodiments, the cell or tissue is a tissue section. Optionally, the tissue section is a cryosection or a fixed tissue section. Optionally, the fixed tissue section is a formalin-fixed tissue section. Optionally, the formalin-fixed tissue section is a formalin-fixed paraffin-embedded (FFPE) tissue section. Optionally, the FFPE tissue section has been treated with xylene to remove paraffin.

In some embodiments, the nucleic acid primer or probe includes a barcode sequence and/or a unique molecular identifier (UMI) sequence.

In embodiments, the nucleic acid primer or probe includes a poly-T sequence.

In certain embodiments, the nucleic acid primer or probe includes a 3′-terminus possessing an enzymatic blocker and at least one RNA base in sufficiently close proximity to the 3′-terminus for a RNase HII enzyme to remove both the enzymatic blocker and the at least one RNA base if the nucleic acid primer or probe specifically anneals with a target nucleic acid molecule, thereby forming a double-stranded substrate for the RNase HII enzyme.

In some embodiments, the composition further includes reverse transcriptase, a DNA polymerase, and/or a RNase HII enzyme.

In embodiments, the cross-linking agent that includes a second monomer or polymer is N,N′-methylene bisacrylamide.

In a related embodiment, the ratio of N,N′-methylene bisacrylamide to acrylamide is about 1:50,000 to about 1:30. Optionally, the ratio of N,N′-methylene bisacrylamide to acrylamide is about 1:40,000 to about 1:100. Optionally, the ratio of N,N′-methylene bisacrylamide to acrylamide is about 1:35,000 to about 1:500. Optionally, the ratio of N,N′-methylene bisacrylamide to acrylamide is about 1:30,000 to about 1:1,000. Optionally, the ratio of N,N′-methylene bisacrylamide to acrylamide is about 1:25,000 to about 1:2,500. Optionally, the ratio of N,N′-methylene bisacrylamide to acrylamide is about 1:20,000 to about 1:5,000. Optionally, the ratio of N,N′-methylene bisacrylamide to acrylamide is about 1:16,667.

In embodiments, the composition further includes tetramethylethylenediamine (TEMED).

In related embodiments, the composition further includes ammonium persulfate (APS) or riboflavin.

Another aspect of the instant disclosure provides a method for binding a target nucleic acid molecule of or associated with a tissue, the method involving: (i) providing a tissue; (ii) contacting the tissue with a first monomer or linear polymer; a cross-linking agent including a second monomer or polymer, where the cross-linking agent is capable of crosslinking with the first monomer or linear polymer when the cross-linking agent and the first monomer or linear polymer are combined; and a nucleic acid primer or probe having a modification capable of binding the primer or probe to the first monomer or linear polymer, to the cross-linking agent, or to both, where the ratio of the cross-linking agent to the first monomer or linear polymer is between about 1:1,000,000 and about 1:30 by weight; (iii) crosslinking the cross-linking agent with the first monomer or linear polymer, thereby forming a matrix; (iv) binding the nucleic acid primer or probe to the first monomer or linear polymer, to the cross-linking agent, or to both; (v) incubating the matrix and nucleic acid primer or probe with the tissue under conditions suitable for annealing of the nucleic acid primer or probe to a target nucleic acid molecule of or associated with the tissue, thereby forming a primer-bound or probe-bound target nucleic acid molecule, and thereby binding a target nucleic acid molecule of or associated with the tissue.

In some embodiments, the method further involves (vi) contacting the primer-bound or probe-bound target nucleic acid molecule with reverse transcriptase, a DNA polymerase, or both.

In certain embodiments, the nucleic acid primer or probe is incubated with the tissue under conditions suitable for amplification of the primer-bound or probe-bound target nucleic acid molecule.

In embodiments, the primer-bound or probe-bound target nucleic acid is bridge amplified. Optionally, bridge amplification is performed in a flow cell.

In certain embodiments, a population of distinct individual target molecules is amplified in situ.

In embodiments, the target molecule is a mRNA.

In some embodiments, the target molecule is a nucleic acid-tagged polypeptide. Optionally, the nucleic acid-tagged polypeptide is a nucleic acid-tagged antibody.

In embodiments, the target nucleic acid is amplified for between five and fifty amplification cycles, or optionally between five and twenty amplification cycles. Optionally, the target nucleic acid is amplified for between ten and fifteen amplification cycles. Optionally, the amplification cycles are bridge amplification cycles.

In some embodiments, the method involves performing one or more cycles of RNase HII and polymerase treatment (e.g., in conditions where the nucleic acid primer or probe includes a 3′-terminus possessing an enzymatic blocker and at least one RNA base in sufficiently close proximity to the 3′-terminus for a RNase HII enzyme to remove both the enzymatic blocker and the at least one RNA base if the nucleic acid primer or probe specifically anneals with a target nucleic acid molecule, thereby forming a double-stranded substrate for the RNase HII enzyme). Optionally, RNase HII and polymerase treatment is performed during exponential bridge amplification. In a related embodiment, RNase HII and polymerase treatment is performed in a single cycle of bridge amplification. In other embodiments, RNase HII and polymerase treatment is performed in 2, 3, 4 or more cycles of bridge amplification treatment. Optionally, the number of bridge amplification cycles that include RNase HII treatment is adjusted to optimize spatial diffusion and signal detection for a given tissue and collection of target sequences. Optionally, additional bridge amplification cycles are performed in the absence of RNase HII.

In certain embodiments, the method further involves contacting the target nucleic acid or an amplicon of the target nucleic acid with a labeled probe. Optionally, the labeled probe is a fluorescently labeled probe.

In embodiments, the target nucleic acid or an amplicon of the target nucleic acid is detected. Optionally, target nucleic acid amplicons are detected with spatial resolution. Optionally, target nucleic acid amplicons are detected with spatial resolution of about 10 μm or less. Optionally, target nucleic acid amplicons are detected with spatial resolution of about 1 μm or less. Optionally, target nucleic acid amplicons are detected with spatial resolution of about 250 nm or less.

In some embodiments, the method further involves sequencing the target nucleic acid or an amplicon of the target nucleic acid in situ. Optionally, the sequencing is sequencing-by-synthesis (SBS).

In certain embodiments, the method further involves detecting the spatial proximity of target nucleic acids by measuring the frequency of recombination events that occur during bridge amplification between amplicons of different target nucleic acids.

In embodiments, the tissue includes neuronal synapses.

In some embodiments, the method further involves determining spatial proximity of two or more target nucleic acids by measuring the frequency of recombination events between amplicons of the two or more target nucleic acids during performance of bridge amplification. Optionally, spatial proximity of the two or more target nucleic acids is detected at a neuronal synapse.

In embodiments, the tissue is fixed with 4% paraformaldehyde (PFA) and/or the tissue is permeabilized with 0.25% Triton.

In certain embodiments, the method further involves bridge amplification of the target nucleic acid in a flow cell at 60° C. Optionally, each cycle of bridge amplification includes a formamide incubation step and a reverse transcriptase polymerization step. Optionally, the bridge amplification is performed for between five and fifty cycles.

In some embodiments, the method further involves contacting bridge-amplified target nucleic acids with primers and reversible 3′ fluorescent nucleotide blockers and performing sequencing-by-synthesis.

In embodiments, the method further involves contacting the matrix with a slide-attached bead array and performing next-generation sequencing (NGS) upon captured target nucleic acids. Optionally, spatial information of the bead array and nucleic acid sequence identities of captured molecules are used to form an image of target nucleic acid distribution, optionally having a spatial resolution of about 50 μm or less. Optionally, spatial resolution is about 10 μm or less. Optionally, spatial resolution is about 1 μm or less. Optionally, spatial resolution is about 250 nm or less.

In certain embodiments, the method further involves forming a puck stack that includes: a first slide; a membrane; the tissue associated with the matrix; and a puck including a bead array attached to a coverslip, where the membrane, tissue section associated with the matrix, and puck including the bead array attached to the coverslip are sandwiched between the first slide and the coverslip, and the tissue section associated with the matrix is sandwiched between the membrane and the puck including the bead array attached to the coverslip.

In a related embodiment, the puck stack of the method further includes a spacer element. Optionally, the puck including the bead array attached to the coverslip, the tissue section associated with the matrix and the membrane are sandwiched between the spacer element and the first slide. Optionally, the spacer element is a paper spacer. Optionally, the paper spacer has a thickness of between about 0.1 and 0.3 mm.

In another embodiment, the puck stack of the method further includes a second slide. Optionally, the puck including the bead array attached to the coverslip, the tissue section associated with the matrix and the membrane are sandwiched between the second slide and the first slide. Optionally, the spacer element is positioned between the second slide and the coverslip and the spacer element, the puck comprising the bead array attached to the coverslip, the tissue section associated with the matrix and the membrane are sandwiched between the second slide and the first slide.

In an additional embodiment, the method further includes performing next-generation sequencing (NGS) upon captured target nucleic acids of the bead array. Optionally, the method involves associating spatial information of the bead array and nucleic acid sequence identities of target nucleic acids to form an image having spatial resolution of about 50 μm or less. Optionally, the method involves associating spatial information of the bead array and nucleic acid sequence identities of target nucleic acids to form an image having spatial resolution of about 10 μm or less. Optionally, the method involves associating spatial information of the bead array and nucleic acid sequence identities of target nucleic acids to form an image having spatial resolution of about 1 μm or less. Optionally, the method involves associating spatial information of the bead array and nucleic acid sequence identities of target nucleic acids to form an image having spatial resolution of about 250 nm or less.

An additional aspect of the instant disclosure provides a kit that includes a composition of the disclosure and instructions for its use.

In certain embodiments, the method further involves forming a puck stack that includes: a first slide; a membrane; the tissue associated with the matrix; and a puck including a bead array attached to a coverslip, where the membrane, tissue section associated with the matrix, and puck including the bead array attached to the coverslip are sandwiched between the first slide and the coverslip, and the tissue section associated with the matrix is sandwiched between the membrane and the puck including the bead array attached to the coverslip.

In a related embodiment, the puck stack of the method further includes a spacer element. Optionally, the puck including the bead array attached to the coverslip, the tissue section associated with the matrix and the membrane are sandwiched between the spacer element and the first slide. Optionally, the spacer element is a paper spacer. Optionally, the paper spacer has a thickness of between about 0.1 and 0.3 mm.

In another embodiment, the puck stack of the method further includes a second slide. Optionally, the puck including the bead array attached to the coverslip, the tissue section associated with the matrix and the membrane are sandwiched between the second slide and the first slide. Optionally, the spacer element is positioned between the second slide and the coverslip and the spacer element, the puck comprising the bead array attached to the coverslip, the tissue section associated with the matrix and the membrane are sandwiched between the second slide and the first slide.

In an additional embodiment, the method further includes performing next-generation sequencing (NGS) upon captured target nucleic acids of the bead array. Optionally, the method involves associating spatial information of the bead array and nucleic acid sequence identities of target nucleic acids to form an image having spatial resolution of about 50 μm or less. Optionally, the method involves associating spatial information of the bead array and nucleic acid sequence identities of target nucleic acids to form an image having spatial resolution of about 10 μm or less. Optionally, the method involves associating spatial information of the bead array and nucleic acid sequence identities of target nucleic acids to form an image having spatial resolution of about 1 μm or less. Optionally, the method involves associating spatial information of the bead array and nucleic acid sequence identities of target nucleic acids to form an image having spatial resolution of about 250 nm or less.

Another aspect of the instant disclosure provides a puck stack, which includes: a first slide; a membrane; a tissue section; and a puck including a bead array attached to a coverslip, where the membrane, tissue section, and puck including the bead array are sandwiched between the first slide and the coverslip, and the tissue section is sandwiched between the membrane and the puck.

In certain embodiments, the puck stack further includes a spacer element. Optionally, the puck including the bead array attached to the coverslip, the tissue section and the membrane are sandwiched between the spacer element and the first slide. Optionally, the spacer element is a paper spacer. Optionally, the paper spacer has a thickness of between about 0.1 and 0.3 mm.

In some embodiments, the puck stack further includes a second slide. Optionally, the puck including the bead array attached to the coverslip, the tissue section and the membrane are sandwiched between the second slide and the first slide. Optionally, the spacer element is positioned between the second slide and the coverslip and the spacer element, the puck including the bead array attached to the coverslip, the tissue section and the membrane are sandwiched between the second slide and the first slide.

In certain embodiments, the tissue section has been processed by the matrix/PONI formation method disclosed herein, thereby forming a primer-bound or probe-bound target nucleic acid molecule and/or matrix associated with the tissue section. Optionally, the primer-bound or probe-bound target nucleic acid molecule associated with the tissue section has been amplified.

A further aspect of the instant disclosure provides a method of processing a puck stack, the method involving: inserting a puck stack of the disclosure into a slide press; apply pressure for a period of time; and creating a compressed puck stack.

In certain embodiments the method further involves removing the puck including the bead array attached to the coverslip from the compressed puck stack.

In related embodiments, the method further involves performing next-generation sequencing (NGS) upon captured target nucleic acids of the bead array. Optionally, the method further involves associating spatial information of the bead array and nucleic acid sequence identities of the target nucleic acids captured by individual beads of the bead array to form an image having spatial resolution of about 50 μm or less. Optionally, the method further involves associating spatial information of the bead array and nucleic acid sequence identities of the target nucleic acids captured by individual beads of the bead array to form an image having spatial resolution of about 10 μm or less. Optionally, the method further involves associating spatial information of the bead array and nucleic acid sequence identities of the target nucleic acids captured by individual beads of the bead array to form an image having spatial resolution of about 1 μm or less. Optionally, the method further involves associating spatial information of the bead array and nucleic acid sequence identities of the target nucleic acids captured by individual beads of the bead array to form an image having spatial resolution of about 250 nm or less.

### Definitions

Unless specifically stated or obvious from context, as used herein, the term “about” is understood as within a range of normal tolerance in the art, for example within 2 standard deviations of the mean. “About” can be understood as within 10%, 9%, 8%, 7%, 6%, 5%, 4%, 3%, 2%, 1%, 0.5%, 0.1%, 0.05%, or 0.01% of the stated value.

In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Unless otherwise clear from context, all numerical values provided herein are modified by the term “about.”

As used herein, the term “amplification,” when used in reference to a nucleic acid, means copying the nucleic acid, wherein the copy has a nucleotide sequence that is the same as or complementary to at least a portion of the nucleotide sequence of the nucleic acid.

As used herein, the term “primer” when used in reference to a nucleic acid means a short nucleic acid sequence that provides a starting point for nucleic acid (e.g., DNA) synthesis. In some embodiments, primers are tagged with barcodes or unique molecular identifiers (UMIs). In some embodiments, primers are added to a pre-matrix solution prior to matrix formation in a cell or tissue. Alternatively, nucleic acid primers can be added to a matrix solution concurrent with or after matrix formation (e.g., during or after cross-linking is performed).

As used herein, the term “amplicon,” when used in reference to a nucleic acid, means the product of copying the nucleic acid, wherein the product has a nucleotide sequence that is the same as or complementary to at least a portion of the nucleotide sequence of the nucleic acid. An amplicon can be produced by any of a variety of amplification methods that use the nucleic acid, or an amplicon thereof, as a template including, for example, bridge amplification, polymerase extension, polymerase chain reaction (PCR), rolling circle amplification (RCA), multiple displacement amplification (MDA), ligation extension, or ligation chain reaction. An amplicon can be a nucleic acid molecule having a single copy of a particular nucleotide sequence (e.g., a PCR product) or multiple copies of the nucleotide sequence (e.g., a recombination product of bridge amplification). A first amplicon of a target nucleic acid is typically a complementary copy. Subsequent amplicons are copies that are created, after generation of the first amplicon, from the target nucleic acid or from the first amplicon. A subsequent amplicon can have a sequence that is substantially complementary to the target nucleic acid or substantially identical to the target nucleic acid.

As used herein, the term “array” refers to a population of features or sites that can be differentiated from each other according to relative location. Different molecules that are at different sites of an array can be differentiated from each other according to the locations of the sites in the array. An individual site of an array can include one or more molecules of a particular type. For example, a site can include a single target nucleic acid molecule having a particular sequence or a site can include several nucleic acid molecules having the same sequence (and/or complementary sequence, thereof).

As used herein, the term “attached” refers to the state of two things being joined, fastened, adhered, connected or bound to each other. For example, an analyte, such as a nucleic acid, can be attached to a material, such as a gel or matrix, by a covalent or non-covalent bond. A covalent bond is characterized by the sharing of pairs of electrons between atoms. A non-covalent bond is a chemical bond that does not involve the sharing of pairs of electrons and can include, for example, hydrogen bonds, ionic bonds, van der Waals forces, hydrophilic interactions and hydrophobic interactions.

As used herein, the term “barcode sequence” is intended to mean a series of nucleotides in a nucleic acid that can be used to identify the nucleic acid, a characteristic of the nucleic acid (e.g., the identity and optionally the location of a bead to which the nucleic acid is attached), or a manipulation that has been carried out on the nucleic acid. In some embodiments the barcode is known as a unique molecular identifier (UMI). The barcode sequence can be a naturally occurring sequence or a sequence that does not occur naturally in the organism from which the barcoded nucleic acid was obtained. A barcode sequence can be unique to a single nucleic acid species in a population or a barcode sequence can be shared by several different nucleic acid species in a population. By way of further example, each nucleic acid probe in a population can include different barcode sequences from all other nucleic acid probes in the population. Alternatively, each nucleic acid probe in a population can include different barcode sequences from some or most other nucleic acid probes in a population. For example, each probe in a population can have a barcode that is present for several different probes in the population even though the probes with the common barcode differ from each other at other sequence regions along their length. In particular embodiments, one or more barcode sequences that are used with a biological specimen (e.g., a tissue sample) are not present in the genome, transcriptome or other nucleic acids of the biological specimen. For example, barcode sequences can have less than 80%, 70%, 60%, 50% or 40% sequence identity to the nucleic acid sequences in a particular biological specimen.

As used herein, the term “bridge amplification,” refers to an amplification method first exemplified in U.S. Ser. No. 12/774,126, which is incorporated herein by reference in its entirety. As employed herein, bridge amplification is a process for the generation of clusters of identical DNA, also referred to herein as “polymerization colonies”, or “PONIs”, to a target of interest. The first stage of bridge amplification involves mixing of one, or more, target nucleic acid molecules under conditions in which primers specific for the target molecules bind to molecules in a pre-matrix solution. For example, a sample (i.e., test sample or tissue sample) can contain a single type of target molecule and the pre-matrix solution can comprise a pair of bound primers specific for that type of target molecule. Alternatively, the sample can contain multiple target molecules and the pre-matrix solution can comprise multiple pairs of bound primers where each pair of primers is specific for one of the target molecules. Or the sample can contain multiple target molecules and the matrix-affixed primers used for bridge amplification are non-specific (e.g., universal, only selective for mRNA amplification, or otherwise) for amplification of bound nucleic acids from the sample (e.g, from the tissue). In embodiments, after adding a pre-matrix solution of the instant disclosure to a cell or tissue and having a matrix form, target molecules of or associated with (e.g., target nucleic acids can include nucleic-acid tagged macromolecules, e.g., polypeptides, e.g, antibodies, that bind polypeptides or other macromolecules present in a target tissue, thereby rendering such target nucleic acids associated with the tissue) the tissue can hybridize with their specific matrix-bound primers. The hybridization complexes that form can then be subjected to amplification, e.g. in an isothermal or thermo-variable flowcell, thus forming double-stranded amplification products. Amplification can include, e.g., from about 10 to about 30 cycles, each cycle including denaturation, primer annealing and polymerization reactions (primer extension) carried out under conditions appropriate for each reaction.

As used herein, the term “flowcell” refers to a glass slide containing small fluidic channels, through which polymerases, dNTPs and buffers can be circulated. Flowcell ambient environments, solutions, and channel design may vary depending on their intended use. In certain embodiments, bridge amplification is performed upon tissue sections within a flowcell.

As used herein, the term “hybridization chain reaction” refers to a chain reaction of hybridization events to form a nicked helix when triggered by a nucleic acid initiator strand. In HCR, short loops of nucleic acids are resistant to invasion by complementary single-stranded nucleic acids. This stability allows for the storage of potential energy in the form of loops; potential energy is released when a triggered conformational change allows the single-stranded bases in the loops to hybridize with a complementary strand. HCR is described in U.S. patent application Ser. No. 11/087,937, filed Mar. 22, 2005, which is incorporated herein by reference.

As used herein, the term “sequencing by synthesis” or “SBS” refers to a method for sequencing nucleic acids, which may be performed in situ. Exemplary SBS procedures, fluidic systems and detection platforms that can be readily adapted for use with a composition, apparatus or method of the present disclosure are described, for example, in Bentley et al., Nature 456:53-59 (2008), PCT Publ. Nos. WO 91/06678, WO 04/018497 or WO 07/123744; U.S. Pat. Nos. 7,057,026, 7,329,492, 7,211,414, 7,315,019 or 7,405,281, and U.S. Patent Publication No. 2008/0108082, each of which is incorporated herein by reference.

As used herein, the term “biological specimen” is intended to mean one or more cell, tissue, organism or portion thereof. A biological specimen can be obtained from any of a variety of organisms. Exemplary organisms include, but are not limited to, a mammal such as a rodent, mouse, rat, rabbit, guinea pig, ungulate, horse, sheep, pig, goat, cow, cat, dog, primate (i.e. human or non-human primate); a plant such as Arabidopsis thaliana, corn, sorghum, oat, wheat, rice, canola, or soybean; an algae such as Chlamydomonas reinhardtii; a nematode such as Caenorhabditis elegans; an insect such as Drosophila melanogaster, mosquito, fruit fly, honey bee or spider; a fish such as zebrafish; a reptile; an amphibian such as a frog or Xenopus laevis; a Dictyostelium discoideum; a fungi such as Pneumocystis carinii, Takifugu rubripes, yeast, Saccharamoyces cerevisiae or Schizosaccharomyces pombe; or a Plasmodium falciparum. Target nucleic acids can also be derived from a prokaryote such as a bacterium, Escherichia coli, Staphylococci or Mycoplasma pneumoniae; an archae; a virus such as Hepatitis C virus or human immunodeficiency virus; or a viroid. Specimens can be derived from a homogeneous culture or population of the above organisms or alternatively from a collection of several different organisms, for example, in a community or ecosystem.

As used herein, the term “cross-linking agent” refers to a molecule capable of bioconjugation to form a branched polymer matrix. “Cross-linking agents” are bifunctional agents containing reactive end groups that respond to functional groups, e.g. primary amines, carboxyls, sulfhydryls and carbonyls. The bifunctional agents are characterized as either homobifunctional or heterobifunctional, allowing for the formation of intermolecular and intramolecular crosslinking. In some embodiments, the cross-linking agent is selected from among the following: polyethylene glycol dimethacrylate, optionally triethylene glycol dimethyacrylate) (TEGDMA) or tetra(ethylene glycol) dimethacrylate, N,N′-methylene bisacrylamide, trisacrylamide, tetracrylamide, amine end-functionalized 4-arm star-PEG, derivatives thereof, and combinations thereof.

As used herein, the terms “monomer” or “linear polymer” when referring to a matrix composition means a precursor to an exogenously derived in situ matrix, optionally where the matrix is cross-linked to a preferred degree (optionally based upon the amount of input crosslinking agent and/or initiator compositions, crosslinking catalysts, or other components). In some embodiments, the monomer or linear polymer is selected from among the following: acrylamide, methacrylate, polyethylene glycol (PEG), carboxymethyl cellulose (CMC), polyvinylpyrrolidone (PVP), isopropylacrylamide, hyaluronic acid, heparin, PLA (polylactic acid), PGA (polyglycolide), and PLGA (poly(lactic-co-glycolic acid)), PHA (Polyhydroxyalkanoates), PPF (propylene fumarate),

agarose, alginate, chitosan, ethylene glycol-decorated polyisocyanide (PIC) polymers, derivatives thereof, and combinations thereof.

As used herein, the term “in situ matrix” refers to a matrix polymerized in situ. In certain embodiments, the in situ matrix is suitable for providing a scaffold for enzymatic reactions. In some embodiments the in situ matrix is both porous and with sufficient structural integrity to covalently bind nucleic acids, e.g., primers or other molecules of interest, while retaining a level of spatial positioning sufficient to allow for spatial positioning of matrix-associated reactions to be obtained at some level of resolution (e.g., 100 μm or less, or other appropriate value of spatial resolution). In some embodiments, a matrix-associated enzymatic reaction is nucleic acid amplification. In some embodiments, the matrix can be polymerized via incubation at a temperature of 4° C. or 37° C., optionally at 4° C. and then 37° C., optionally repeating the temperature incubation steps 1, 2, 3, 4, or 5 times, optionally adding an initiator composition, optionally where the initiator composition is ammonium persulfate (APS) and tetramethylethylenediamine (TEMED), optionally wherein the initiator composition is riboflavin and TEMED.

As used herein, the term “porosity” when referring to a matrix composition refers to a measure of the void (i.e. “empty”) spaces in a material, and is a fraction of the volume of voids over the total volume, between 0 and 1, or as a percentage between 0% and 100%. In some embodiments, an in situ matrix is referred to as “porous” if it permits the passage of enzymes necessary for nucleic acid bridge amplification.

As used herein, the term “spatial proximity information” refers to the relative spatial relationship of two molecules. In some embodiments, the two molecules are tagged with barcodes. In some exemplary embodiments, spatial proximity information is recorded through amplicons combining with neighboring sequences during bridge amplification. The closer the two sequences, the more likely they are to be recombined on the same amplicon. As described in Weinstein et al. (DNA Microscopy: Optics-free Spatio-genetic Imaging by a Stand-Alone Chemical Reaction. Cell. vol 178(1) 2019), an algorithm decodes molecular proximities from the recombined sequences and infers physical images of the original transcripts at cellular resolution with precise sequence information. Spatial proximity information may be determined for PONIs using this method in any tissue, with an exemplary embodiment being detecting macromolecule spatial proximities in the vicinity of individual synapses in situ.

By “control” or “reference” is meant a standard of comparison. Methods to select and test control samples are within the ability of those in the art. Determination of statistical significance is within the ability of those skilled in the art, e.g., the number of standard deviations from the mean that constitute a positive result.

As used herein, the term “cryosection” refers to a piece of tissue, e.g. a biopsy, that has been obtained from a subject, snap frozen, embedded in optimal cutting temperature embedding material, frozen, and cut into thin sections. In certain embodiments, the thin sections can be fixed and permeabilized prior to adding a matrix-forming solution, in which a branched polymer with bound amplification primers polymerizes in situ.

As used herein, the term “different,” when used in reference to nucleic acids, means that the nucleic acids have nucleotide sequences that are not the same as each other. Two or more nucleic acids can have nucleotide sequences that are different along their entire length. Alternatively, two or more nucleic acids can have nucleotide sequences that are different along a substantial portion of their length. For example, two or more nucleic acids can have target nucleotide sequence portions that are different for the two or more molecules while also having a universal sequence portion that is the same on the two or more molecules.

As used herein, the term “each,” when used in reference to a collection of items, is intended to identify an individual item in the collection but does not necessarily refer to every item in the collection. Exceptions can occur if explicit disclosure or context clearly dictates otherwise.

As used herein, the term “extend,” or “polymerize” when used in reference to a nucleic acid, is intended to mean addition of at least one nucleotide or oligonucleotide to the nucleic acid. In particular embodiments one or more nucleotides can be added to the 3′ end of a nucleic acid, for example, via polymerase catalysis (e.g. DNA polymerase, RNA polymerase or reverse transcriptase). Chemical or enzymatic methods can be used to add one or more nucleotide to the 3′ or 5′ end of a nucleic acid. One or more oligonucleotides can be added to the 3′ or 5′ end of a nucleic acid, for example, via chemical or enzymatic (e.g. ligase catalysis) methods. A nucleic acid can be extended in a template directed manner, whereby the product of extension is complementary to a template nucleic acid that is hybridized to the nucleic acid that is extended.

As used herein, the term “next-generation sequencing” or “NGS” can refer to sequencing technologies that have the capacity to sequence polynucleotides at speeds that were unprecedented using conventional sequencing methods (e.g., standard Sanger or Maxam-Gilbert sequencing methods). In some embodiments, NGS is performed after in situ bridge amplification PONIs are released from the tissue. The unprecedented speeds of NGS are achieved by performing and reading out thousands to millions of sequencing reactions in parallel. NGS sequencing platforms include, but are not limited to, the following: Massively Parallel Signature Sequencing (Lynx Therapeutics); 454 pyro-sequencing (454 Life Sciences/Roche Diagnostics); solid-phase, reversible dye-terminator sequencing (Solexa/Illumina™); SOLiD™ technology (Applied Biosystems); Ion semiconductor sequencing (Ion Torrent™); and DNA nanoball sequencing (Complete Genomics). Descriptions of certain NGS platforms can be found in the following: Shendure, et al., “Next-generation DNA sequencing,” Nature, 2008, vol. 26, No. 10, 135-1 145; Mardis, “The impact of next-generation sequencing technology on genetics,” Trends in Genetics, 2007, vol. 24, No. 3, pp. 133-141; Su, et al., “Next-generation sequencing and its applications in molecular diagnostics” Expert Rev Mol Diagn, 2011, 11 (3):333-43; and Zhang et al., “The impact of next-generation sequencing on genomics”, J Genet Genomics, 201, 38(3): 95-109.

As used herein, the terms “nucleic acid” and “nucleotide” are intended to be consistent with their use in the art and to include naturally occurring species or functional analogs thereof. Particularly useful functional analogs of nucleic acids are capable of hybridizing to a nucleic acid in a sequence specific fashion or capable of being used as a template for replication of a particular nucleotide sequence.

Naturally occurring nucleic acids generally have a backbone containing phosphodiester bonds. An analog structure can have an alternate backbone linkage including any of a variety of those known in the art. Naturally occurring nucleic acids generally have a deoxyribose sugar (e.g. found in deoxyribonucleic acid (DNA)) or a ribose sugar (e.g. found in ribonucleic acid (RNA)). A nucleic acid can contain nucleotides having any of a variety of analogs of these sugar moieties that are known in the art. A nucleic acid can include native or non-native nucleotides. In this regard, a native deoxyribonucleic acid can have one or more bases selected from the group consisting of adenine, thymine, cytosine or guanine and a ribonucleic acid can have one or more bases selected from the group consisting of uracil, adenine, cytosine or guanine. Useful non-native bases that can be included in a nucleic acid or nucleotide are known in the art. The terms “probe” or “target,” when used in reference to a nucleic acid or sequence of a nucleic acid, are intended as semantic identifiers for the nucleic acid or sequence in the context of a method or composition set forth herein and does not necessarily limit the structure or function of the nucleic acid or sequence beyond what is otherwise explicitly indicated. The terms “probe” and “target” can be similarly applied to other analytes such as proteins, small molecules, cells or the like.

In certain embodiments, an oligonucleotide probe or primer of the instant disclosure includes a blocking moiety, e.g., an enzymatic blocker, e.g., a moiety that blocks or is capable of blocking polymerase activity. Any blocker moiety capable of significantly inhibiting polymerase-mediated extension at the 3′-terminus of an oligonucleotide is contemplated for use in rhPCR assay embodiments disclosed herein. Exemplary 3′-terminal enzymatic blockers include, without limitation, dideoxy nucleotides (i.e., ddG, ddA, ddT, ddC, and analogs thereof), C3 propanediol spacers, and other C3 blocking modifications.

In certain embodiments, an oligonucleotide of the disclosure includes one or more ribonucleotides (RNA), optionally in sufficient proximity to the 3′-terminus of the oligonucleotide to allow for RNase HII to cleave the oligonucleotide at the RNA when target nucleic acid is annealed to the oligonucleotide. It is expressly contemplated that any nucleotide or nucleotide analog that is cleavable by RNase HII could successfully substitute for the one or more RNAs in the oligonucleotides of the instant disclosure. Examples of sufficient proximity of the one or more RNAs to the 3′-terminus of the oligonucleotide include having the one or more RNA residues positioned within an oligonucleotide immediately 5′ of a region of 2, 3, 4, 5 or 6 (or optionally more residues, provided that RNase HII-mediated RNA cleavage can still occur) nucleotides that extend the region of complementarity of the oligonucleotide to a target nucleic acid, which are then followed by a 3′-terminal enzymatic blocker (e.g., a 3′-terminal dideoxy nucleotide or other C3 blocking moiety at the 3′-terminal residue of the oligonucleotide). In some embodiments, oligonucleotides are employed for rhPCR, with impact of enhancing both specificity and sensitivity, even allowing for, e.g., allele-specific discrimination between target nucleic acid sequences with high specificity and sensitivity.

As used herein, the term “subject” includes humans and mammals (e.g., mice, rats, pigs, cats, dogs, and horses). In many embodiments, subjects are mammals, particularly primates, especially humans. In some embodiments, subjects are livestock such as cattle, sheep, goats, cows, swine, and the like; poultry such as chickens, ducks, geese, turkeys, and the like; and domesticated animals particularly pets such as dogs and cats. In some embodiments (e.g., particularly in research contexts) subject mammals will be, for example, rodents (e.g., mice, rats, hamsters), rabbits, primates, or swine such as inbred pigs and the like.

As used herein, the term “tissue” is intended to mean an aggregation of cells, and, optionally, intercellular matter. Typically, the cells in a tissue are not free floating in solution and instead are attached to each other to form a multicellular structure. Exemplary tissue types include muscle, nerve, epidermal and connective tissues.

As used herein, the term “universal sequence” refers to a series of nucleotides that is common to two or more nucleic acid molecules even if the molecules also have regions of sequence that differ from each other. A universal sequence that is present in different members of a collection of molecules can allow capture of multiple different nucleic acids using a population of universal capture nucleic acids that are complementary to the universal sequence. Similarly, a universal sequence present in different members of a collection of molecules can allow the replication or amplification of multiple different nucleic acids using a population of universal primers that are complementary to the universal sequence. Thus, a universal capture nucleic acid or a universal primer includes a sequence that can hybridize specifically to a universal sequence. Target nucleic acid molecules may be modified to attach universal adapters, for example, at one or both ends of the different target sequences.

Unless specifically stated or obvious from context, as used herein, the term “or” is understood to be inclusive. Unless specifically stated or obvious from context, as used herein, the terms “a”, “an”, and “the” are understood to be singular or plural.

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it is understood that the particular value forms another aspect. It is further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. It is also understood that throughout the application, data are provided in a number of different formats and that this data represent endpoints and starting points and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 as well as all intervening decimal values between the aforementioned integers such as, for example, 1.1, 1.2, 1.3, 1.4, 1.5, 1.6, 1.7, 1.8, and 1.9. With respect to sub-ranges, “nested sub-ranges” that extend from either end point of the range are specifically contemplated. For example, a nested sub-range of an exemplary range of 1 to 50 may comprise 1 to 10, 1 to 20, 1 to 30, and 1 to 40 in one direction, or 50 to 40, 50 to 30, 50 to 20, and 50 to 10 in the other direction.

The transitional term “comprising,” which is synonymous with “including,” “containing,” or “characterized by,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention.

The embodiments set forth below and recited in the claims can be understood in view of the above definitions.

Other features and advantages of the disclosure will be apparent from the following description of the preferred embodiments thereof, and from the claims. Unless otherwise defined, all technical and scientific terms used herein have the same meaning as commonly understood by one of ordinary skill in the art to which this disclosure belongs. Although methods and materials similar or equivalent to those described herein can be used in the practice or testing of the present disclosure, suitable methods and materials are described below. All published foreign patents and patent applications cited herein are incorporated herein by reference. All other published references, documents, manuscripts and scientific literature cited herein are incorporated herein by reference. In the case of conflict, the present specification, including definitions, will control. In addition, the materials, methods, and examples are illustrative only and not intended to be limiting.

### BRIEF DESCRIPTION OF THE DRAWINGS

The following detailed description, given by way of example, but not intended to limit the disclosure solely to the specific embodiments described, may best be understood in conjunction with the accompanying drawings, in which:

FIGS. 1A-1D depict schematics showing the steps of bridge amplification in situ in brain tissue. FIG. 1A depicts a cross-section of brain tissue that has been fixed and permeabilized. FIG. 1B depicts an exemplary in situ matrix of the instant disclosure, e.g., a low bis-acrylamide matrix, in which amplification primers with a 5′ acrydite modification were combined with the matrix solution, providing a covalently-bound primer array for formation of polymerization colonies in situ (PONIs). FIG. 1C depicts generation of cDNAs complementary to primer-bound endogenous target RNAs, using the acrylamide-bound primers and a reverse transcriptase. FIG. 1D depicts further amplification of the cDNA array, using bridge amplification, to generate PONIs.

FIG. 2 depicts an exemplary use for the primer-bound matrix of the instant disclosure, e.g. in situ sequencing performed via sequencing-by-synthesis upon captured target nucleic acids and their amplicons (i.e. PONIs).

FIG. 3 shows HPCA (hippocalcin) mRNA expression detected via in situ bridge amplification using a primer-bound matrix composition of the instant disclosure. Left panels show 20× and 60× images of a control experiment in which no amplification primers were used. Right panels show 20× and 60× images of bridge-amplified HPCA. The HPCA PONIs were labeled with fluorescent probes using in situ DNA-hybridization chain reaction (HCR). Scale bars are 25 μm.

FIG. 4 shows that in situ bridge amplification as disclosed herein maintained spatial integrity. Left panels show MBP (myelin basic protein) mRNA expression detected via in situ bridge amplification, at far left, and endogenous in situ hybridization (ISH) in a reference tissue section, at second from left. Arrows indicate corresponding regions. Right panels show HPCA mRNA expression detected via in situ bridge amplification, at far right, and endogenous in situ hybridization (ISH) in a reference tissue section, at second from right. Arrows indicate corresponding regions. The HPCA PONIs were labeled with fluorescent probes using in situ DNA-hybridization chain reaction (HCR).

FIG. 5 shows HPCA mRNA expression detected via in situ bridge amplification using matrix solutions having different percentages of bis-acrylamide to total acrylamide in solution. Tissues shown were from the “CA1” region (the first hippocampal circuit), the “slm” region (stratum lacunosum-moleculare), and the “DG” region (dentate gyrus). As demonstrated, the optimal percentage of bis-acrylamide to total acrylamide in solution for these exemplary experiments was found to be 1.5×10⁻⁴ to 1.5×10⁻³. The HPCA PONIs were labeled with fluorescent probes using in situ DNA-hybridization chain reaction (HCR).

FIG. 6 demonstrates that PONIs increased in both count and size with additional bridge amplification cycles. At left is a negative control panel, in which no amplification primers were used. The middle panel shows detection of HPCA mRNA in situ bridge amplification where 10 bridge amplification cycles were performed. The right panel shows detection of HPCA mRNA in situ bridge amplification where 15 bridge amplification cycles were performed. For detection, HPCA PONIs were labeled with fluorescent probes using in situ DNA-hybridization chain reaction (HCR).

FIGS. 7A and 7B depict another exemplary application of the in situ matrices of the instant disclosure, in which spatial proximity information is obtained by measuring the frequency of individual amplicons combining with neighboring sequences during bridge amplification, which produces recombined amplicons at a rate proportionate to the proximity of the recombined target sequences/amplicons. The closer the two nucleic acid sequences are, the more likely they are to be found on the same recombined amplicon obtained via bridge sequencing. FIG. 7A depicts the recombination of two nearby DNA sequences during bridge amplification. FIG. 7B depicts relationships between the spatial proximity of barcode-containing amplicons and the number of recombination events observed during bridge amplification.

FIGS. 8A and 8B show unique molecular identifier (UMI) counts obtained in assessing position and abundance of oligonucleotide-conjugated antibodies in PONI-processed tissue using the hybridization and PONI detection methods of the instant disclosure. FIG. 8A respectively shows the number of anti-CD200 antibody UMIs and control antibody UMIs identified in an experiment designed to assess the signal-to-noise of immunolabeling in PONI-processed tissue. Notably, the UMI counts of anti-CD200 antibody were vastly greater than the UMI counts for the control antibody, thereby demonstrating that the anti-CD200 antibody retained specificity after all of the enzymatic processes involved in PONI. FIG. 8B shows UMI counts of recombination events that were also identified for each antibody (anti-CD200 antibody and control antibody). Notably, the UMI count for anti-CD200 antibody-RNA recombination was greater than the UMI count for the anti-CD200 antibody (˜3× fold), which demonstrated the ability for an individual molecule to recombine with multiple distinct neighboring molecules during the bridge amplification/PONI process.

FIGS. 9A and 9B depict plots of anti-CD200 antibody recombination-enriched genes, as compared to CD200 gene expression in mouse thalamus. FIG. 9A shows genes enriched in the anti-CD200 antibody recombination dataset, as compared to total cDNA. Red represents the top 15 genes enriched in the anti-CD200 antibody recombination dataset. Green represents the top 15 genes under-enriched in the anti-CD200 antibody recombination dataset. Yellow represents overlaps between the red and green data points. Genes were plotted onto a single-cell dataset from a mouse thalamus. FIG. 9B shows CD200 gene expression in a single-cell dataset from a mouse thalamus. Notably, the anti-CD200 antibody recombination-enriched gene set almost completely matched the expression of CD200.

FIGS. 10A and 10B show schematics of an embodiment in which PONI primers contain an enzymatic blocker at the 3′ end and a single RNA base in proximity to the 3′ end. FIG. 10A shows a schematic in which PONI primers anneal to a cDNA strand with an incomplete match of sequence, causing incomplete annealing between primer and target nucleic acid strands and a scenario in which RNase HII is incapable of cleaving the PONI primer at the RNA base. The enzymatic blocker therefore remains at the 3′ end of the PONI primer, preventing primer extension from occurring. FIG. 10B conversely shows a schematic in which PONI primers anneal to a target nucleic acid (cDNA strand) with a complete match of sequence, thereby allowing RNase HII to cleave the PONI primer at the RNA base, removing the 3′-terminal enzymatic blocker and thereby liberating the resultant 3′-terminus of the RNase HII-cleaved PONI primer for polymerase-mediated extension.

FIGS. 11A and 11B depict a schematic of embodiments in which one or more RNase HII treatment and polymerization bridge amplification (PONT) cycles are employed. FIG. 11A shows how users can elect to perform only a low number of RNase HII bridge amplification cycles, thereby limiting cDNA (target nucleic acid) diffusion and thus limiting recombination events to a small range. FIG. 11B shows how, conversely, users can elect to perform a high number of RNase HII bridge amplification cycles, thereby allowing for greater amounts of cDNA (target nucleic acid) diffusion and expanding the recombination range. Control and flexibility in the interaction range(s) of interest is thereby achieved.

FIGS. 12A, 12B and 12C depict the compatibility of the currently disclosed in situ hybridization and detection methods with the previously disclosed “Slide-seq” in situ transcriptome abundance measurement and imaging platform. FIG. 12A shows a schematic of a method developed herein to apply PONI-processed tissue to a Slide-seq detection array (puck). Tissue sections were mounted on porous polyester track etch (PETE) membranes and processed using a PONI whole-transcriptome amplification protocol before being placed on a Slide-seq puck. FIG. 12B shows Slide-seq in situ transcript abundance data for two transcripts (Hpca and Mbp) in PONI-amplified tissue that was then applied to a SlideSeq puck. FIG. 12C shows, for comparison, in situ hybridization images for Hpca and Mbp, respectively, in mouse brain tissue sections found in the Allen Brain Atlas. This comparison between the Slide-seq and ISH data revealed a great consistency in the spatial distribution of both Hpca and Mbp, thereby demonstrating the instant PONI approach's compatibility with SlideSeq.

FIGS. 13A, 13B and 13C depict a sandwich protocol employed with PONI-processed tissue sections of the instant disclosure. FIG. 13A shows a cross-sectional view of an initial sandwich prior to compression. FIG. 13B shows a cross-sectional view of a sandwich after compression. FIG. 13C shows a cross-sectional view of a portion of a compressed sandwich for use in Slide-seq analysis.

### DETAILED DESCRIPTION OF THE INVENTION

The present disclosure is directed, at least in part, to the discovery that targeted amplification of nucleic acids in tissue (e.g., coding and non-coding RNAs; nuclear/genomic DNA; mtDNA; pathogen nucleic acids; and single cell, forensic, and paleoarcheology uses, etc.) can be performed efficiently and precisely when a porous in situ matrix, e.g., an acrylamide matrix having a low ratio of bis-acrylamide/acrylamide, is polymerized in proximity to a tissue sample (or is polymerized and then contacted with a tissue sample). The instant disclosure therefore provides compositions and methods for in situ nucleic acid amplification where the precise characterization of a tissue-endogenous or tissue-associated nucleic acid's amount, localization, and sequence may be determined in situ, with high levels of spatial resolution (e.g., resolutions of 10 μm or less as currently exemplified). Contemplated applications for such improved in situ amplification compositions and methods include, without limitation, measurement of coding and non-coding RNA sequences, including their amounts and spatial position/distribution; measurement of amounts and spatial position/distribution of nucleic acid-tagged macromolecules (e.g., nucleic acid-tagged antibodies); spatial relationships between macromolecules; assessment of copy number variation (CNV); mitochondrial lineage tracing; assessment of epigenetic regulation; identification of regions of monoallelic gene expression and gene dosage in an assayed tissue; evaluation of nucleic acid therapy deliverables to tissue, including, e.g., identification of cellular delivery of RNAi, CRISPR/Cas9 plasmid(s) and/or gels, TALEN plasmid(s) and/or gels, viral vectors (e.g., AAV), and delivery of expression vectors/plasmids in general; among others.

As exemplified, the current disclosure employs bridge amplification, as described in U.S. Ser. No. 12/774,126, for amplification of nucleic acids associated with an in situ matrix. Certain exemplified embodiments include in situ sequencing and/or detection of spatial proximity between individual molecules—both endogenous nucleic acids or nucleic acid-tagged moieties (e.g., nucleic acid-tagged macromolecules capable of binding a target macromolecule (e.g., protein, nucleic acid or other macromolecule) present in or associated with a tissue). Currently, rolling circle amplification or combinatorial fluorescent hybridization probes are most commonly used for nucleic acid detection in situ. However, using hybridization probes requires a time-intensive protocol, and cannot be done genome-wide. Furthermore, the hybridization probe approach typically requires a large sequence to be available per target of interest. Rolling circle amplification has numerous disadvantages, including inefficiency of production, and inability to modify each amplicon copy (e.g. with primers), or to control the amount of amplification. Creating PONIs (polymerization colonies or Polonies In situ), as demonstrated herein, provides superior control of amplification, increased density of the sequence(s) of interest, and the ability to modify each amplicon (such as tagging each amplicon with a UMI or recombining amplicons with each other in a specific fashion).

The instant disclosure importantly provides for and enables application of bridge amplification to a tissue sample. The instant discovery of a bis-acrylamide/acrylamide matrix that remains porous to enzymes and its application to in situ nucleic acid amplification and detection is a key advance of the instant disclosure. In exemplified in situ matrix solutions, and acrylamide monomer solution was employed and contained a very low bis-acrylamide to acrylamide ratio (in one exemplary embodiment, about 1:30,000). When polymerized in tissue, the in situ matrix provided better structural integrity than linear polyacrylamide for fixed oligonucleotides while still maintaining sufficient porosity to permit the passage of enzymes into the tissue sample.

The instant disclosure therefore provides an improved platform for in situ sequencing, spatial resolution, and/or detection of spatial proximity between individual molecules. In each case, the instant disclosure provides for the direct investigation of nucleic acids or of nucleic acid-tagged moieties, such as nucleic acid-tagged antibodies designed to bind a protein or population of proteins of interest. In all currently exemplified cases, PONIs are created via in situ bridge amplification, which is enabled by the exemplary low-bis acrylamide matrix. To create PONIs from RNA, cDNA is produced via reverse transcription prior to polymerization. To create polonies of nucleic acid-tagged moieties, the moieties (such as antibodies or probes against selected endogenous RNA or DNA sequences) can be applied to the tissue to tag the molecules of interest before polymerization. Once PONIs are formed, in situ sequencing can optionally be conducted through sequencing by synthesis. Spatial proximity can also be assessed by bridge amplifying target nucleic acids using primers containing overlapping overhangs. In this way, nearby amplicons are able to recombine with each other as they further amplify. The newly recombined molecule will then contain sequences of both amplicons. Information regarding the frequency of such recombinant-forming events can then be obtained downstream via sequencing, thereby allowing for determination of which molecules were within a certain spatial distance of one other. Because the rate of these recombination events should decrease as a function of spatial distance, the frequency of recombination events will be inversely correlated to the distance between the molecules.

Various expressly contemplated components of certain compositions and methods of the instant disclosure are considered in additional detail below.

Detecting the spatial distribution of macromolecules in tissue samples is important for many studies. Certain compositions and methods of the instant disclosure have been exemplified to obtain high resolution spatial information regarding macromolecule distribution(s) in tissue samples, including specifically in brain tissue. Further applications of the compositions and methods of the instant disclosure are also envisioned, as there are numerous situations in which the precise characterization of a macromolecule's (or a population of macromolecules') abundance, localization, and/or identity (e.g., sequence for nucleic acid target(s)) can prove helpful. In addition to genomic, research, drug development and drug delivery uses, it is noted that forensic and paleoarcheology work can be severely limited by nucleic acid sample size, implicating additional applications of the compositions and methods of the current disclosure. It is also well accepted that molecular analysis determination of genomic instability in various pathological condition such as cancer, is most precisely carried out in well-defined cell populations, such as that obtained by laser capture micro dissection or cell sorting.

Likewise, the ability to amplify ribonucleic acid (RNA) is an important aspect of efforts to elucidate biological processes. mRNA represents gene expression activity at a defined time. Non-coding RNAs have been shown to be of great importance in regulation of various cellular functions and in certain disease pathologies. Such RNAs are often present at very low levels in tissues. Thus, the compositions and amplification methods of the instant disclosure, which are capable of amplifying both high and low abundance RNAs, determining the abundance of individual RNAs, as well as the spatial positioning of a target RNA at sub-cellular resolution, provides a further important advance.

The compositions and methods of the instant disclosure therefore provide an advance over certain known approaches for in situ amplification, sequencing, and detection, examples of which include STARmap (spatially-resolved transcript amplicon readout mapping) (Wang X. et al. Three-dimensional intact-tissue sequencing of single-cell transcriptional states. Science vol. 361(6400), 2018), MERFISH (Chen et al. Spatially resolved, highly multiplexed RNA profiling in single cells. Science vol. 348 (6233), 2015), BaristaSeq (Chen et al. Efficient in situ barcode sequencing using padlock probe-based BaristaSeq. Nucleic acids research vol. 46(4), 2018), and IGS (In situ genome sequencing) (Payne et al. In situ genome sequencing resolves DNA sequence and structure in intact biological samples. Science, Dec. 31, 2020). DNA Microscopy, an optics-free imaging process in which macromolecules are labeled and their position is detected through nucleic acid amplification, is another art-recognized method related to nucleic acid amplification and detection.

Additional details of the instant disclosure are provided in the following sections.

### In Situ Matrix Components and Preparation

Matrices of the instant disclosure can be formed from any of a variety of matrix-forming monomers or polymers known in the art. Exemplary matrices include a monomer or linear component and a branched component (crosslinking agent), though matrices that include only branch-forming components are also known in the art and can be employed herein. In certain embodiments, the in situ matrix is suitable for providing a scaffold for enzymatic reactions. In some embodiments the in situ matrix is both porous and with sufficient structural integrity to covalently bind nucleic acids, e.g., primers or other molecules of interest, while retaining a level of spatial positioning sufficient to allow for spatial positioning of matrix-associated reactions to be obtained at some level of resolution (e.g., 100 μm or less, or other appropriate value of spatial resolution). In some embodiments, a matrix-associated enzymatic reaction is nucleic acid amplification. In some embodiments, the matrix is cross-linked to a preferred degree (optionally based upon the amount of input crosslinking agent and/or initiator compositions, crosslinking catalysts, or other components). In some embodiments, the monomer or linear polymer is acrylamide, methacrylate, polyethylene glycol (PEG), carboxymethyl cellulose (CMC), polyvinylpyrrolidone (PVP), isopropylacrylamide, hyaluronic acid, heparin, PLA (polylactic acid), PGA (polyglycolide), and PLGA (poly(lactic-co-glycolic acid)), PHA (Polyhydroxyalkanoates), PPF (propylene fumarate), agarose, alginate, chitosan, or ethylene glycol-decorated polyisocyanide (PIC) polymers, derivatives thereof, and combinations thereof. In some embodiments, the cross-linking agent is polyethylene glycol dimethacrylate (optionally triethylene glycol dimethyacrylate (TEGDMA) or tetra(ethylene glycol) dimethacrylate), N,N′-methylene bisacrylamide, trisacrylamide, tetracrylamide, or amine end-functionalized 4-arm star-PEG, derivatives thereof, or combinations thereof. It is also contemplated that sufficiently rigid yet porous matrices for purpose of the instant disclosure can be formed from individual monomers or polymers of any of the preceding monomers or polymers, or by individual polymerizable/cross-linkable components known in the art. In some embodiments, a matrix of the instant disclosure can be polymerized via incubation at a temperature of 4° C. or 37° C., optionally at 4° C. and then 37° C., optionally repeating the temperature incubation steps 1, 2, 3, 4, or 5 times, optionally adding an initiator composition, optionally where the initiator composition is ammonium persulfate (APS) and tetramethylethylenediamine (TEMED), optionally where the initiator composition is riboflavin and TEMED.

In some embodiments, the ratio of the cross-linking agent to the first monomer or linear polymer is at most 1:50 by weight, in some embodiments, the ratio of the cross-linking agent to the monomer or linear polymer is at most 1:100 by weight, in some embodiments, the ratio of the cross-linking agent to the monomer or linear polymer is at most 1:500 by weight, in some embodiments, the ratio of the cross-linking agent to the monomer or linear polymer is at most 1:1,000 by weight, in some embodiments, the ratio of the cross-linking agent to the monomer or linear polymer is at most 1:2,000 by weight, in some embodiments, the ratio of the cross-linking agent to the monomer or linear polymer is at most 1:3,000 by weight, in some embodiments, the ratio of the cross-linking agent to the monomer or linear polymer is at most 1:5,000 by weight, in some embodiments, the ratio of the cross-linking agent to the monomer or linear polymer is at most 1:10,000 by weight, in some embodiments, the ratio of the cross-linking agent to the monomer or linear polymer is at most 1:20,000 by weight, in some embodiments, the ratio of the cross-linking agent to the monomer or linear polymer is at most 1:30,000 by weight, in some embodiments, the ratio of the cross-linking agent to the monomer or linear polymer is at most 1:40,000 by weight, in some embodiments, the ratio of the cross-linking agent to the monomer or linear polymer is at most 1:50,000 by weight, in some embodiments, the ratio of the cross-linking agent to the monomer or linear polymer is at most 1:75,000 by weight, in some embodiments, the ratio of the cross-linking agent to the monomer or linear polymer is at most 1:100,000 by weight, in some embodiments, the ratio of the cross-linking agent to the monomer or linear polymer is at most 1:200,000 by weight, in some embodiments, the ratio of the cross-linking agent to the monomer or linear polymer is at most 1:300,000 by weight, in some embodiments, the ratio of the cross-linking agent to the monomer or linear polymer is at most 1:400,000 by weight, in some embodiments, the ratio of the cross-linking agent to the monomer or linear polymer is at most 1:500,000 by weight, in some embodiments, ratio of the cross-linking agent to the monomer or linear polymer is at most 1:600,000 by weight, in some embodiments, the ratio of the cross-linking agent to the to the monomer or linear polymer is at most 1:700,000, in some embodiments, the cross-linking agent to the to the monomer or linear polymer is at most 1:800,000, in some embodiments, the ratio of the cross-linking agent to the to the monomer or linear polymer is at most 1:900,000, in some embodiments, the ratio of the cross-linking agent to the to the monomer or linear polymer is at most 1:1,000,000.

### Matrix-Associated Nucleic Acid Primers and Probes

Certain aspects of the instant disclosure feature matrix-associated nucleic acid primers or probes, which are used for capture of target nucleic acids, and optionally for amplification in situ. Association of a nucleic acid primer or probe with a matrix component and/or matrix can be performed by art-recognized means, the most common of which employ modified nucleic acid primers or probes to achieve such associations. Exemplary nucleic acid modifications that can be employed to attach a nucleic acid primer or probe to a matrix component and/or matrix include, without limitation, acrydite, biotin-streptavidin, magnetic beads, digoxigenin (DIG), PEG, nanoparticles, peptides, antigens for the purpose of binding an antibody, and related molecules that allow for the initial binding and subsequent polymerization of nucleic acids. In some embodiments, a nucleic acid modification comprising free COOH groups can be activated to become reactive to amine functional groups in a matrix, and vice versa. In some cases, an acrydite moiety can refer to an acrydite analogue generated from the reaction of acrydite with one or more species, such as, for example, the reaction of acrydite with other monomers and cross-linkers during a polymerization reaction. Acrydite moieties may be modified to form chemical bonds with a species to be attached, such as an oligonucleotide. For example, acrydite moieties may be modified with thiol groups capable of forming a disulfide bond or may be modified with groups already having a disulfide bond. The thiol or disulfide may be used as an anchor point for a species to be attached or another part of the acrydite moiety may be used for attachment. In some cases, attachment is reversible, such that when the disulfide bond is broken (e.g., in the presence of a reducing agent), the agent is released from the matrix or other support. In other cases, an acrydite moiety includes a reactive hydroxyl group that may be used for attachment.

### Tissue Samples and Sectioning

In some embodiments, a tissue section is employed. The tissue can be derived from a multicellular organism. Exemplary multicellular organisms include, but are not limited to a mammal, plant, algae, nematode, insect, fish, reptile, amphibian, fungi or Plasmodium falciparum. Exemplary species are set forth previously herein or known in the art. The tissue can be freshly excised from an organism or it may have been previously preserved for example by freezing, embedding in a material such as paraffin (e.g. formalin fixed paraffin embedded samples), formalin fixation, infiltration, dehydration or the like. Optionally, a tissue section can be cryosectioned, using techniques and compositions as described herein and as known in the art. As a further option, a tissue can be permeabilized and the cells of the tissue lysed. Any of a variety of art-recognized lysis treatments can be used. Target nucleic acids that are released from a tissue that is permeabilized can be captured by nucleic acid probes, as described herein and as known in the art.

A tissue can be prepared in any convenient or desired way for its use in a method, composition or apparatus herein. Fresh, frozen, fixed or unfixed tissues can be used. A tissue can be fixed or embedded using methods described herein or known in the art.

A tissue sample for use herein, can be fixed by deep freezing at temperature suitable to maintain or preserve the integrity of the tissue structure, e.g. less than −20° C. A fixed or embedded tissue sample can be sectioned, i.e. thinly sliced, using known methods. For example, a tissue sample can be sectioned using a chilled microtome or cryostat, set at a temperature suitable to maintain both the structural integrity of the tissue sample and the chemical properties of the nucleic acids in the sample. Exemplary additional fixatives that are expressly contemplated include alcohol fixation (e.g., methanol fixation, ethanol fixation), glutaraldehyde fixation and paraformaldehyde fixation.

In some embodiments, a tissue sample will be treated to remove embedding material (e.g. to remove paraffin or formalin) from the sample prior to release, capture or modification of nucleic acids. This can be achieved by contacting the sample with an appropriate solvent (e.g. xylene and ethanol washes).

A particularly relevant source for a tissue sample is a human being. The sample can be derived from an organ, including for example, an organ of the central nervous system such as brain, brainstem, cerebellum, spinal cord, cranial nerve, or spinal nerve; an organ of the musculoskeletal system such as muscle, bone, tendon or ligament; an organ of the digestive system such as salivary gland, pharynx, esophagus, stomach, small intestine, large intestine, liver, gallbladder or pancreas; an organ of the respiratory system such as larynx, trachea, bronchi, lungs or diaphragm; an organ of the urinary system such as kidney, ureter, bladder or urethra; a reproductive organ such as ovary, fallopian tube, uterus, vagina, placenta, testicle, epididymis, vas deferens, seminal vesicle, prostate, penis or scrotum; an organ of the endocrine system such as pituitary gland, pineal gland, thyroid gland, parathyroid gland, or adrenal gland; an organ of the circulatory system such as heart, artery, vein or capillary; an organ of the lymphatic system such as lymphatic vessel, lymph node, bone marrow, thymus or spleen; a sensory organ such as eye, ear, nose, or tongue; or an organ of the integument such as skin, subcutaneous tissue or mammary gland. In some embodiments, a tissue sample is obtained from a bodily fluid or excreta such as blood, lymph, tears, sweat, saliva, semen, vaginal secretion, ear wax, fecal matter or urine.

A sample from a human can be considered (or suspected) healthy or diseased when used. In some cases, two samples can be used: a first being considered diseased and a second being considered as healthy (e.g. for use as a healthy control). Any of a variety of conditions can be evaluated, including but not limited to, cancer, an autoimmune disease, cystic fibrosis, aneuploidy, pathogenic infection, psychological condition, hepatitis, diabetes, sexually transmitted disease, heart disease, stroke, cardiovascular disease, multiple sclerosis or muscular dystrophy. Certain contemplated conditions include genetic conditions or conditions associated with pathogens having identifiable mRNA transcript signatures.

### Permeabilizing Agents

Certain aspects of the instant disclosure feature permeabilizing agents, examples of which tend to compromise and/or remove the protective boundary of lipids often surrounding cellular macromolecules. Disruption of cellular lipid barriers via administration of a permeabilizing agent can provide enhanced physical access to cellular macromolecules, such as DNA, RNA, or proteins, that might otherwise be relatively inaccessible. Specifically contemplated examples of permeabilizing agents include, without limitation: Triton X-100, NP-40, methanol, acetone, Tween 20, saponin, Leucoperm™, and digitonin, among others.

### Nucleosome Disrupting Agents

In some embodiments of the instant disclosure, chromatin structure is disrupted to allow for greater access to chromatin regions that might otherwise be inaccessible/under-represented, thereby providing improved genomic representation of assayed DNA molecules in such regions. As exemplified herein, nucleosomes can be disrupted via contact with HCl, SDS and/or a protease/proteinase.

### Nucleic Acid Probe Annealing, Amplification and Sequencing of Target Nucleic Acids

Certain aspects of the instant disclosure feature nucleic acid primers or probes that are designed to anneal target nucleic acids in or associated with a contacted tissue. A primer is a short nucleic acid sequence that provides a starting point for DNA synthesis. In some embodiments, nucleic acid primers are tagged with barcodes or unique molecular identifiers (UMIs). A “barcode sequence” is a series of nucleotides in a nucleic acid that can be used to identify the nucleic acid, a characteristic of the nucleic acid, or a manipulation that has been carried out on the nucleic acid. In some embodiments the barcode is known as a unique molecular identifier (UMI). The barcode sequence can be a naturally occurring sequence or a sequence that does not occur naturally in the organism from which the barcoded nucleic acid was obtained. A barcode sequence can be unique to a single nucleic acid species in a population or a barcode sequence can be shared by several different nucleic acid species in a population. By way of further example, each nucleic acid probe in a population can include different barcode sequences from all other nucleic acid probes in the population. Alternatively, each nucleic acid probe in a population can include different barcode sequences from some or most other nucleic acid probes in a population. For example, each probe in a population can have a barcode that is present for several different probes in the population even though the probes with the common barcode differ from each other at other sequence regions along their length. In particular embodiments, one or more barcode sequences that are used with a biological specimen (e.g., a tissue sample) are not present in the genome, transcriptome or other nucleic acids of the biological specimen. For example, barcode sequences can have less than 80%, 70%, 60%, 50% or 40% sequence identity to the nucleic acid sequences in a particular biological specimen.

A nucleic acid probe hybridizes to single-stranded nucleic acid (DNA or RNA) whose base sequence allows probe-target base pairing due to complementarity between the probe and target. The labeled probe is first denatured into single stranded DNA (ssDNA) and then hybridized to the target ssDNA or ssRNA immobilized in situ, e.g., in a matrix or other solid support. The probe is tagged or “labeled” to detect hybridization of the probe to its target sequence. In some embodiments, fluorescent hybridization probes may be used to detect and localize DNA and/or RNA sequences to define the spatial-temporal patterns of gene expression within cells and tissues. In some embodiments, the probe may be a poly-T probe for binding a population of mRNAs and detecting mRNA levels within an annealed population of target mRNA molecules.

In some embodiments, attachment of a nucleic acid probe is non-specific with regard to any sequence differences between the nucleic acid probe and other nucleic acid probes that are or will be attached to a matrix. For example, different probes can have a universal sequence that complements matrix-attached primers or the different probes can have a common moiety that mediates attachment to the matrix. Alternatively, each of the different probes (or a subpopulation of different probes) can have a unique (or sufficiently unique) sequence that complements a unique (or sufficiently unique) primer bound to the matrix or they can have a unique (or sufficiently unique) moiety that interacts with one or more different reactive moieties in the matrix. In such cases, the unique (or sufficiently unique) primers or unique (or sufficiently unique) moieties can, optionally, be attached at predefined locations in order to selectively capture particular probes, or particular types of probes, at the respective predefined locations.

Nucleic acid probes that are used in a method set forth herein or present in an apparatus or composition of the present disclosure can include barcode sequences, and for embodiments that include a plurality of different nucleic acid probes, each of the probes can include a different barcode sequence from other probes in the plurality. Barcode sequences can be any of a variety of lengths.

Longer sequences can generally accommodate a larger number and variety of barcodes for a population. Generally, all probes in a plurality will have the same length barcode (albeit with different sequences), but it is also possible to use different length barcodes for different probes. A barcode sequence can be at least 2, 4, 6, 8, 10, 12, 15, 20 or more nucleotides in length. Alternatively, or additionally, the length of the barcode sequence can be at most 20, 15, 12, 10, 8, 6, 4 or fewer nucleotides. Examples of barcode sequences that can be used are set forth, for example, in U.S. Patent Publication No. 2014/0342921 and U.S. Pat. No. 8,460,865, each of which is incorporated herein by reference.

Sequencing techniques, such as sequencing-by-synthesis (SBS) techniques, are a useful method for determining barcode sequences in situ. SBS can be carried out as follows. To initiate a first SBS cycle, one or more labeled nucleotides, DNA polymerase, SBS primers etc., can be contacted with one or more features in a tissue or cell (e.g. feature(s) where nucleic acid probes are attached to a matrix). Those features where SBS primer extension causes a labeled nucleotide to be incorporated can be detected. Optionally, the nucleotides can include a reversible termination moiety that terminates further primer extension once a nucleotide has been added to the SBS primer. For example, a nucleotide analog having a reversible terminator moiety can be added to a primer such that subsequent extension cannot occur until a deblocking agent is delivered to remove the moiety. Thus, for embodiments that use reversible termination, a deblocking reagent can be delivered to the matrix (before or after detection occurs). Washes can be carried out between the various delivery steps. The cycle can then be repeated n times to extend the primer by n nucleotides, thereby detecting a sequence of length n. Exemplary SBS procedures, fluidic systems and detection platforms that can be readily adapted for use with a composition, apparatus or method of the present disclosure are described, for example, in Bentley et al., Nature 456:53-59 (2008), PCT Publ. Nos. WO 91/06678, WO 04/018497 or WO 07/123744; U.S. Pat. Nos. 7,057,026, 7,329,492, 7,211,414, 7,315,019 or 7,405,281, and U.S. Patent Publication No. 2008/0108082, each of which is incorporated herein by reference.

Other sequencing procedures, wherein in some embodiments, the PONIs are released from the tissue include the use of cyclic reactions, such as pyrosequencing. Pyrosequencing detects the release of inorganic pyrophosphate (PPi) as particular nucleotides are incorporated into a nascent nucleic acid strand (Ronaghi, et al., Analytical Biochemistry 242(1), 84-9 (1996); Ronaghi, Genome Res. 1 1 (1), 3-1 1 (2001); Ronaghi et al. Science 281 (5375), 363 (1998); or U.S. Pat. Nos. 6,210,891, 6,258,568 or 6,274,320, each of which is incorporated herein by reference). In pyrosequencing, released PPi can be detected by being immediately converted to adenosine triphosphate (ATP) by ATP sulfurylase, and the level of ATP generated can be detected via luciferase-produced photons. Thus, the sequencing reaction can be monitored via a luminescence detection system.

Excitation radiation sources used for fluorescence-based detection systems are not necessary for pyrosequencing procedures. Useful fluidic systems, detectors and procedures that can be used for application of pyrosequencing to apparatus, compositions or methods of the present disclosure are described, for example, in PCT Patent Publication No. WO2012/058096, US Patent Publication No. 2005/0191698 A1, or U.S. Pat. Nos. 7,595,883 or 7,244,559, each of which is incorporated herein by reference.

Sequencing-by-ligation reactions are also useful, wherein in some embodiments PONIs are released from the tissue, including, for example, those described in Shendure et al. Science 309:1728-1732 (2005); or U.S. Pat. Nos. 5,599,675 or 5,750,341, each of which is incorporated herein by reference. Some embodiments can include sequencing-by-hybridization procedures as described, for example, in Bains et al., Journal of Theoretical Biology 135(3), 303-7 (1988); Drmanac et al., Nature Biotechnology 16, 54-58 (1998); Fodor et al., Science 251 (4995), 767-773 (1995); or PCT Publication No. WO 1989/10977, each of which is incorporated herein by reference. In both sequencing-by-ligation and sequencing-by-hybridization procedures, target nucleic acids (or amplicons thereof) that are present at sites of an array are subjected to repeated cycles of oligonucleotide delivery and detection. Compositions, apparatus or methods set forth herein or in references cited herein can be readily adapted for sequencing-by-ligation or sequencing-by-hybridization procedures. Typically, the oligonucleotides are fluorescently labeled and can be detected using fluorescence detectors similar to those described with regard to SBS procedures herein or in references cited herein.

Some sequencing embodiments wherein PONIs are released from the tissue, can utilize methods involving the real-time monitoring of DNA polymerase activity. For example, nucleotide incorporations can be detected through fluorescence resonance energy transfer (FRET) interactions between a fluorophore-bearing polymerase and γ-phosphate-labeled nucleotides, or with zeromode waveguides (ZMWs). Techniques and reagents for FRET-based sequencing are described, for example, in Levene et al. Science 299, 682-686 (2003); Lundquist et al. Opt. Lett. 33, 1026-1028 (2008); and Korlach et al. Proc. Natl. Acad. Sci. USA 105, 1 176-1 181 (2008), each of which is incorporated herein by reference.

Some sequencing embodiments, wherein PONIs are released from the tissue, include detection of a proton released upon incorporation of a nucleotide into an extension product. For example, sequencing based on detection of released protons can use an electrical detector and associated techniques that are commercially available from Ion Torrent (Guilford, CT, a Life Technologies and Thermo Fisher subsidiary) or sequencing methods and systems described in U.S. Patent Publication Nos. 2009/0026082 A1; 2009/0127589 A1; 2010/0137143 A1; or U.S. Publication No. 2010/0282617 A1, each of which is incorporated herein by reference.

Nucleic acid hybridization techniques are also useful methods for determining barcodes both in situ and ex situ. In some embodiments, methods utilize labelled nucleic acid decoder probes that are complementary to at least a portion of a barcode sequence. In some cases, pools of many different probes with distinguishable labels are used, thereby allowing a multiplex decoding operation. The number of different barcodes determined in a decoding operation can exceed the number of labels used for the decoding operation. For example, decoding can be carried out in several stages where each stage constitutes hybridization with a different pool of decoder probes. The same decoder probes can be present in different pools but the label that is present on each decoder probe can differ from pool to pool (i.e. each decoder probe is in a different “state” when in different pools).

Various combinations of these states and stages can be used to expand the number of barcodes that can be decoded well beyond the number of distinct labels available for decoding. Such combinatorial methods are set forth in further detail in U.S. Pat. No. 8,460,865 or Gunderson et al., Genome Research 14:870-877 (2004), each of which is incorporated herein by reference.

A method of the present disclosure can include a step of contacting a biological specimen (i.e., a sectioned tissue sample in which nucleic acid sequence targets of interest have been amplified through bridge amplification, wherein PONIs are formed) with a matrix that has nucleic acid probes attached thereto, as described in PCT/US19/30194. In some embodiments, the nucleic acid probes are randomly located on matrix. The identity and location of the nucleic acid probes may have been decoded prior to contacting the biological specimen with the matrix.

A nucleic acid probe used in a composition or method set forth herein can include a target capture moiety. In particular embodiments, the target capture moiety is a target capture sequence. The target capture sequence is generally complementary to a target sequence such that target capture occurs by formation of a probe-target hybrid complex. A target capture sequence can be any of a variety of lengths including, for example, lengths exemplified above in the context of barcode sequences.

In certain embodiments, a plurality of different nucleic acid probes can include different target capture sequences that hybridize to different target nucleic acid sequences from a biological specimen. Different target capture sequences can be used to selectively bind to one or more desired target

All or part of a target nucleic acid that is hybridized to a nucleic acid probe can be copied by extension. For example, an extended probe can include at least, 1, 2, 5, 10, 25, 50, 100, 200, 500, 1000 or more nucleotides that are copied from a target nucleic acid. The length of the extension product can be controlled, for example, using reversibly terminated nucleotides in the extension reaction and running a limited number of extension cycles. The cycles can be run as exemplified for SBS techniques and the use of labeled nucleotides is not necessary.

Modified nucleic acid probes (e.g. extended nucleic acid probes) that are released from an in situ matrix can be pooled to form a fluidic mixture. The mixture can include, for example, at least 10, 100, 1×10³, 1×10⁴, 1×10⁵, 1×10⁶, 1×10⁷, 1×10⁸, 1×10⁹ or more different modified probes. Alternatively or additionally, a fluidic mixture can include at most 1×10⁹, 1×10⁸, 1×10⁷, 1×10⁶, 1×10⁵, 1×10⁴, 1×10³, 100, 10 or fewer different modified probes. The fluidic mixture can be manipulated to allow detection of the modified nucleic acid probes. For example, the modified nucleic acid probes can be separated spatially on a second solid support (i.e., different from the in situ matrix from which the nucleic acid probes were released after having been contacted with a biological specimen and modified), or the probes can be separated temporally in a fluid stream.

Modified nucleic acid probes (e.g. extended nucleic acid probes) can be separated on a bead or other solid support in a capture or detection method commonly employed for microarray-based techniques or nucleic acid sequencing techniques such as those set forth previously. For example, modified probes can be attached to a microarray by hybridization to complementary nucleic acids. The modified probes can be attached to beads or to a flow cell surface and optionally undergo additional rounds of amplification as is carried out in many nucleic acid sequencing platforms. Modified probes can be separated in a fluid stream using a microfluidic device, droplet manipulation device, or flow cytometer. Typically, detection is carried out on these separation devices, but detection is not necessary in all embodiments.

It is further expressly contemplated that in addition to the above-described sequence features, oligonucleotides of the instant disclosure can possess any number of other art-recognized features while remaining within the scope of the instant disclosure.

### In Situ Sequencing

In certain aspects of the disclosure, in situ sequencing is performed by any art-recognized mode of parallel (optionally massively parallel) in situ sequencing, examples of which particularly include the previously described SOLiD™ method, which is a sequencing-by-ligation technique that can be performed in situ upon a solid support (refer, e.g., to Voelkerding et al, Clinical Chem., 55-641-658, 2009; U.S. Pat. Nos. 5,912,148; and 6,130,073, which are incorporated herein by reference in their entireties). In certain embodiments of the instant disclosure, such sequencing can be performed upon a PONI array in an in situ matrix present on a standard microscope slide, optionally using a standard microscope fitted with sufficient computing power to track and associate individual sequences during progressive rounds of detection, with their spatial position(s). The instant disclosure also employed custom fluidics, incubation times, enzymatic mixes and imaging setup in performing in situ sequencing.

Integration with “Slide-Seq” Arrays

In certain embodiments, it is expressly contemplated that matrix-associated captured target nucleic acids and/or amplicons thereof can not only be identified and resolved via performance of in situ methods such as in situ sequencing, but can also be identified and resolved using approaches that retain spatial information of contacted surfaces (e.g., tissues and/or the in situ matrix of the current disclosure) via use of tagged arrays that retain sequence information while NGS sequencing is performed. An exemplary such approach that can readily be used in association with the currently disclosed compositions and methods is the “Slide-seq” approach of PCT/US19/30194, which enabled RNA capture from tissue with high resolution. In an exemplary application, a matrix of the current disclosure having probe-attached target nucleic acids and/or amplicons (e.g., obtained from a tissue) can be contacted with a “Slide-seq” array (i.e. a slide-attached bead array with known and/or resolvable spatial tags) and NGS sequencing can be performed upon the target nucleic acids and/or amplicons that have transferred to the “Slide-seq” array. Using such a combination of methods, the high throughput advantages of NGS sequencing can be applied to the compositions and methods of the instant disclosure, while retaining high resolution spatial information.

### Other Sequencing Methods

Some of the methods and compositions provided herein employ methods of sequencing nucleic acids. A number of DNA sequencing techniques are known in the art, including fluorescence-based sequencing methodologies (See, e.g., Birren et al, Genome Analysis Analyzing DNA, 1, Cold Spring Harbor, N.Y., which is incorporated herein by reference in its entirety). In some embodiments, automated sequencing techniques understood in that art are utilized. In some embodiments, parallel sequencing of partitioned amplicons can be utilized (PCT Publication No WO2006084132, which is incorporated herein by reference in its entirety). In some embodiments, DNA sequencing is achieved by parallel oligonucleotide extension (See, e.g., U.S. Pat. Nos. 5,750,341; 6,306,597, which are incorporated herein by reference in their entireties). Additional examples of sequencing techniques include the Church polony technology (Mitra et al, 2003, Analytical Biochemistry 320, 55-65; Shendure et al, 2005 Science 309, 1728-1732; U.S. Pat. Nos. 6,432,360, 6,485,944, 6,511,803, which are incorporated by reference), the 454 picotiter pyrosequencing technology (Margulies et al, 2005 Nature 437, 376-380; US 20050130173, which are incorporated herein by reference in their entireties), the Solexa single base addition technology (Bennett et al, 2005, Pharmacogenomics, 6, 373-382; U.S. Pat. Nos. 6,787,308; 6,833,246, which are incorporated herein by reference in their entireties), the Lynx massively parallel signature sequencing technology (Brenner et al. (2000). Nat. Biotechnol. 18:630-634; U.S. Pat. Nos. 5,695,934; 5,714,330, which are incorporated herein by reference in their entireties), and the Adessi PCR colony technology (Adessi et al. (2000). Nucleic Acid Res. 28, E87; WO 00018957, which are incorporated herein by reference in their entireties).

Next-generation sequencing (NGS) methods can be employed in certain aspects of the instant disclosure to obtain a high volume of sequence information (such as are particularly required to perform deep sequencing of mRNA generated PONIs in a highly efficient and cost effective manner. NGS methods share the common feature of massively parallel, high-throughput strategies, with the goal of lower costs in comparison to older sequencing methods (see, e.g., Voelkerding et al, Clinical Chem., 55: 641-658, 2009; MacLean et al, Nature Rev. Microbiol, 7-287-296; which are incorporated herein by reference in their entireties). NGS methods can be broadly divided into those that typically use template amplification and those that do not. Amplification-utilizing methods include pyrosequencing commercialized by Roche as the 454 technology platforms (e.g., GS 20 and GS FLX), the Solexa platform commercialized by Illumina, and the Supported Oligonucleotide Ligation and Detection (SOLiD™) platform commercialized by Applied Biosystems. Non-amplification approaches, also known as single-molecule sequencing, are exemplified by the Heli Scope platform commercialized by Helicos Biosciences, SMRT sequencing commercialized by Pacific Biosciences, and emerging platforms marketed by VisiGen and Oxford Nanopore Technologies Ltd.

In the Solexa/Illumina platform (Voelkerding et al, Clinical Chem., 55-641-658, 2009; MacLean et al, Nature Rev. Microbiol, 7:287-296; U.S. Pat. Nos. 6,833,246; 7,115,400; 6,969,488, which are incorporated herein by reference in their entireties), sequencing data are produced in the form of shorter-length reads. In this method, single-stranded fragmented DNA is end-repaired to generate 5′-phosphorylated blunt ends, followed by Klenow-mediated addition of a single A base to the 3′ end of the fragments. A-addition facilitates addition of T-overhang adaptor oligonucleotides, which are subsequently used to capture the template-adaptor molecules on the surface of a flow cell that is studded with oligonucleotide anchors. The anchor is used as a PCR primer, but because of the length of the template and its proximity to other nearby anchor oligonucleotides, extension by PCR results in the “arching over” of the molecule to hybridize with an adjacent anchor oligonucleotide to form a bridge structure on the surface of the flow cell. These loops of DNA are denatured and cleaved. Forward strands are then sequenced with reversible dye terminators. The sequence of incorporated nucleotides is determined by detection of post-incorporation fluorescence, with each fluorophore and block removed prior to the next cycle of dNTP addition. Sequence read length ranges from 36 nucleotides to over 50 nucleotides, with overall output exceeding 1 billion nucleotide pairs per analytical run.

Sequencing nucleic acid molecules using SOLiD technology (Voelkerding et al, Clinical Chem., 55: 641-658, 2009; U.S. Pat. Nos. 5,912,148; and 6,130,073, which are incorporated herein by reference in their entireties) can initially involve fragmentation of the template, ligation to oligonucleotide adaptors, and clonal amplification by emulsion PCR. Following this, templates are immobilized on a derivatized surface of a glass flow-cell, and a primer complementary to the adaptor oligonucleotide is annealed. However, rather than utilizing this primer for 3′ extension, it is instead used to provide a 5′ phosphate group for ligation to interrogation probes containing two probe-specific bases followed by 6 degenerate bases and one of four fluorescent labels. In the SOLiD system, interrogation probes have 16 possible combinations of the two bases at the 3′ end of each probe, and one of four fluors at the 5′ end. Fluor color, and thus identity of each probe, corresponds to specified color-space coding schemes. Multiple rounds (usually 7) of probe annealing, ligation, and fluor detection are followed by denaturation, and then a second round of sequencing using a primer that is offset by one base relative to the initial primer. In this manner, the template sequence can be computationally re-constructed, and template bases are interrogated twice, resulting in increased accuracy. Sequence read length averages 35 nucleotides, and overall output exceeds 4 billion bases per sequencing run.

In certain embodiments, nanopore sequencing is employed (see, e.g., Astier et al, J. Am. Chem. Soc. 2006 Feb. 8; 128(5): 1705-10, which is incorporated by reference). The theory behind nanopore sequencing has to do with what occurs when a nanopore is immersed in a conducting fluid and a potential (voltage) is applied across it. Under these conditions a slight electric current due to conduction of ions through the nanopore can be observed, and the amount of current is exceedingly sensitive to the size of the nanopore. As each base of a nucleic acid passes through the nanopore (or as individual nucleotides pass through the nanopore in the case of exonuclease-based techniques), this causes a change in the magnitude of the current through the nanopore that is distinct for each of the four bases, thereby allowing the sequence of the DNA molecule to be determined.

The Ion Torrent technology is a method of DNA sequencing based on the detection of hydrogen ions that are released during the polymerization of DNA (see, e.g., Science 327(5970): 1190 (2010); U.S. Pat. Appl. Pub. Nos. 20090026082, 20090127589, 20100301398, 20100197507, 20100188073, and 20100137143, which are incorporated herein by reference in their entireties). A microwell contains a template DNA strand to be sequenced. Beneath the layer of microwells is a hypersensitive ISFET ion sensor. All layers are contained within a CMOS semiconductor chip, similar to that used in the electronics industry. When a dNTP is incorporated into the growing complementary strand a hydrogen ion is released, which triggers a hypersensitive ion sensor. If homopolymer repeats are present in the template sequence, multiple dNTP molecules will be incorporated in a single cycle. This leads to a corresponding number of released hydrogens and a proportionally higher electronic signal. This technology differs from other sequencing technologies in that no modified nucleotides or optics are used. The per base accuracy of the Ion Torrent sequencer is approximately 99.6% for 50 base reads, with approximately 100 Mb generated per run. The read-length is 100 base pairs. The accuracy for homopolymer repeats of 5 repeats in length is approximately 98%. The benefits of ion semiconductor sequencing are rapid sequencing speed and low upfront and operating costs.

### Imaging/Image Assembly

In certain embodiments, the spatial locations of a large number of amplicons (including barcoded amplicons) within an array can first be assigned to an image location, with all associated nucleic acid sequence data also assigned to that position. High resolution images representing the extent of capture of individual or grouped nucleic acid sequences across the various spatial positions of the in situ matrix can then be generated using the underlying sequence information. Images (i.e., pixel coloring and/or intensities) can be adjusted and/or normalized using any (or any number of) art-recognized technique(s) deemed appropriate by one of ordinary skill in the art.

In certain embodiments, a high-resolution image of the instant disclosure is an image in which discrete features (e.g., pixels) of the image are spaced at 50 μm or less. In some embodiments, the spacing of discrete features within the image is at 40 μm or less, optionally 30 μm or less, optionally 20 μm or less, optionally 15 μm or less, optionally 10 μm or less, optionally 9 μm or less, optionally 8 μm or less, optionally 7 μm or less, optionally 6 μm or less, optionally 5 μm or less, optionally 4 μm or less, optionally 3 μm or less, optionally 2 μm or less, or optionally 1 μm or less.

Images can be obtained using detection devices known in the art. Examples include microscopes configured for light, bright field, dark field, phase contrast, fluorescence, reflection, interference, or confocal imaging. A biological specimen can be stained prior to imaging to provide contrast between different regions or cells. In some embodiments, more than one stain can be used to image different aspects of the specimen (e.g. different regions of a tissue, different cells, specific subcellular components or the like). In other embodiments, a biological specimen can be imaged without staining.

In particular embodiments, a fluorescence microscope (e.g. a confocal fluorescent microscope) can be used to detect a biological specimen that is fluorescent, for example, by virtue of a fluorescent label. Fluorescent specimens can also be imaged using a nucleic acid sequencing device having optics for fluorescent detection such as a Genome Analyzer®, MiSeq®, NextSeq® or HiSeq® platform device commercialized by Illumina, Inc. (San Diego, CA); or a SOLiD™ sequencing platform commercialized by Life Technologies (Carlsbad, CA). Other imaging optics that can be used include those that are found in the detection devices described in Bentley et al., Nature 456:53-59 (2008), PCT Publ. Nos. WO 91/06678, WO 04/018497 or WO 07/123744; U.S. Pat. Nos. 7,057,026, 7,329,492, 7,211,414, 7,315,019 or 7,405,281, and US Pat. App. Publ. No. 2008/0108082, each of which is incorporated herein by reference.

An image of a biological specimen can be obtained at a desired resolution, for example, to distinguish tissues, cells or subcellular components. Accordingly, the resolution can be sufficient to distinguish components of a biological specimen that are separated by at least 0.5 μm, 1 μm, 5 μm, 10 μm, 50 μm, 100 μm, 500 μm, 1 mm or more. Alternatively or additionally, the resolution can be set to distinguish components of a biological specimen that are separated by at least 1 mm, 500 μm, 100 μm, 50 μm, 10 μm, 5 μm, 1 μm, 0.5 μm or less.

### Kits

The instant disclosure also provides kits containing agents of this disclosure for use in the methods of the present disclosure. Kits of the instant disclosure may include one or more containers. In some embodiments, the kits further include instructions for use in accordance with the methods of this disclosure. In some embodiments, these instructions comprise a description of administration of the agent to diagnose, e.g., a disease and/or malignancy. In some embodiments, the instructions comprise a description of how to create a tissue cryosection, treat a tissue section with a forward and reverse amplification primers; matrix precursor monomers or linear polymers; a cross-linking agent; a reverse transcriptase; a flow cell to perform bridge amplification and generate polonies in situ (PONIs); sequencing primers and reversible 3′ fluorescent nucleotide blockers to sequence the PONIs by synthesis; and instructions for use. The kit may further comprise a description of selecting an individual suitable for treatment based on identifying whether that subject has a certain pattern of nucleic acid amplification, sequence and/or localization of one or more nucleic acid sequences in a cryosection sample.

Instructions supplied in the kits of the instant disclosure are typically written instructions on a label or package insert (e.g., a paper sheet included in the kit), but machine-readable instructions (e.g., instructions carried on a magnetic or optical storage disk) are also acceptable.

The label or package insert indicates that the composition is used for staging a cryosection and/or diagnosing a specific amplitude, sequence, and/or localization pattern in a cryosection. Instructions may be provided for practicing any of the methods described herein.

The kits of this disclosure are in suitable packaging. Suitable packaging includes, but is not limited to, vials, bottles, jars, flexible packaging (e.g., sealed Mylar or plastic bags), and the like. The container may further comprise a pharmaceutically active agent.

Kits may optionally provide additional components such as buffers and interpretive information. Normally, the kit comprises a container and a label or package insert(s) on or associated with the container.

The practice of the present disclosure employs, unless otherwise indicated, conventional techniques of chemistry, molecular biology, microbiology, recombinant DNA, genetics, immunology, cell biology, cell culture and transgenic biology, which are within the skill of the art. See, e.g., Maniatis et al., 1982, Molecular Cloning (Cold Spring Harbor Laboratory Press, Cold Spring Harbor, N.Y.); Sambrook et al., 1989, Molecular Cloning, 2nd Ed. (Cold Spring Harbor Laboratory Press, Cold Spring Harbor, N.Y.); Sambrook and Russell, 2001, Molecular Cloning, 3rd Ed. (Cold Spring Harbor Laboratory Press, Cold Spring Harbor, N.Y.); Ausubel et al., 1992), Current Protocols in Molecular Biology (John Wiley & Sons, including periodic updates); Glover, 1985, DNA Cloning (IRL Press, Oxford); Anand, 1992; Guthrie and Fink, 1991; Harlow and Lane, 1988, Antibodies, (Cold Spring Harbor Laboratory Press, Cold Spring Harbor, N.Y.); Jakoby and Pastan, 1979; Nucleic Acid Hybridization (B. D. Hames & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Methods In Enzymology, Vols. 154 and 155 (Wu et al. eds.), Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); Riott, Essential Immunology, 6th Edition, Blackwell Scientific Publications, Oxford, 1988; Hogan et al., Manipulating the Mouse Embryo, (Cold Spring Harbor Laboratory Press, Cold Spring Harbor, N.Y., 1986); Westerfield, M., The zebrafish book. A guide for the laboratory use of zebrafish (Danio rerio), (4th Ed., Univ. of Oregon Press, Eugene, 2000).

Unless otherwise defined, all technical and scientific terms used herein have the same meaning as commonly understood by one of ordinary skill in the art to which this disclosure belongs. Although methods and materials similar or equivalent to those described herein can be used in the practice or testing of the present disclosure, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Reference will now be made in detail to exemplary embodiments of the disclosure. While the disclosure will be described in conjunction with the exemplary embodiments, it will be understood that it is not intended to limit the disclosure to those embodiments. To the contrary, it is intended to cover alternatives, modifications, and equivalents as may be included within the spirit and scope of the disclosure as defined by the appended claims. Standard techniques well known in the art or the techniques specifically described below were utilized.

### EXAMPLES

### Example 1: Materials and Methods

### Tissue Preparation

Tissues were fixed and permeabilized, prior to addition of in situ matrix solution, with paraformaldehyde (PFA) and Triton X-100. Cryosections of brain tissue were fixed for 10 minutes with 4% PFA/PBS at room temperature; washed 3× with PBS; permeabilized for 10 minutes with 0.25% Triton/PBS at room temperature; washed 3× with PBS; permeabilized for 5 minutes with 0.1 N HCl at room temperature; and washed an additional 3× with PBS. Endogenous tissue DNA was degraded using DNase (deoxyribonuclease) by applying the DNase mix below and incubating for 2 hours at 25° C. 5.0 μl of 15 mM EDTA was later added and the tissue was washed 3× with PBS for 5 minutes each at room temperature. Reverse transcription of endogenous RNA in the tissue was performed by addition of the RT Mix below, incubating for at least 15 minutes at 25° C., and subsequently incubating overnight at 37° C.

DNase Mix: 30 μl total − 30 μl per 9 mm gasket

1.5 μl of DNase I with a stock @ 10 U/μl
0.5 μl of RNase Inhibitor
3 μl of 10x DNase I Buffer
25 μl of ddH20

RT mix: 50 μl total − 50 μl per 9 mm gasket

2.5 μl of SuperScript IV
2 μl of DTT (Stock @ 0.1M)
1.25 μl of dNTP Mix (Stock @ 10 mM)
10 μl of 5x SSIV RT Bμffer
1.25 μl of RT primer (100 μM)
0.5 μl of RNase Inhibitor
33 μl of ddH2O

### In Situ Matrix Solution and PONI Primer Addition

To form an in situ matrix solution that remained relatively aqueous but also capable of covalently binding amplification primers in situ, a low ratio of bis-acrylamide/acrylamide (1:16,667; 1.7×10⁻⁴%:5%) was used. Prior to addition of the in situ matrix solution, tissues were washed 3× with PBS at room temperature in a gasket (9×9 mm, Frame-Seal Slide Chambers obtained from Bio-Rad™). 25 μl/well of the below bis-acrylamide/acrylamide in situ matrix solution (also containing the below acrylamide monomer solution as detailed) was added to each well, and the gasket was incubated for 5 minutes at 4° C. The first aliquot of in situ matrix solution was then removed and a second 25 μl/well in situ matrix solution was added to each well and incubated for 25 minutes at 4° C. A parafilm-wrapped slide was placed on the gasket, ensuring that there were no bubbles, and the composition was incubated for 2 hours at 37° C. Finally, the tissues were washed 3× with excess PBS.

Endogenous RNA was then degraded by applying the below RNase Mix, incubating for 2 hours at 37° C., and washing 3× with PBS.

25% Acrylamide Monomer Solution

1 ml total - Aliquot and store in −20° C.
500 μl Acrylamide (Stock is 50 g/100 ml)
25 μl Tris Base (Stock is 1M)
475 μl ddH2O

In situ matrix Solution (5% PONI Matrix), keep on ice until use

60 μl total -enough for 2 wells
12 μl 25% Acrylamide Solution
0.6 μl TEMED (Stock @ 10% in H2O)
0.6 μl APS (Stock @ 10% in H2O) - added immediately prior to
application
9 μl 0.002% Bis-acrylamide
6 μl FWD Primer (2 mM stock) (5′ acrydite label)
6 μl REV Primer (2 mM stock) (5′ acrydite label)
25.8 μl UP Water

RNase Mix

30 μl total - 30 μl per 9 mm gasket
3 μl Roche RNase Blend (10 mg/ml)
0.75 μl RNase H (10 U/ul)
3 μl NEB 4 Buffer
23.25 μl ddH2O

### Flowcell Bridge Amplification

Flowcells were used to form PONIs in situ through bridge amplification. Bridge amplification has previously been performed in vitro, as described in U.S. Ser. No. 12/774,126. The tissues contacted with in situ matrix solution and PONI primers were incubated for 15 minutes with an HCR wash buffer without heparin at 37° C. prior to bridge amplification. The polymerase mix below was added to the flow cell, and the flowcell was run with the following parameters: formamide time 2 minutes, water wash, polymerase time 5 minutes, and for 5-50 cycles.

Polymerase Mix ~25 ml total-~15 cycles

2.5 ml Isothermal Amplification Buffer
2.5 ml dNTP Mix (2 mM)
150 μl MgSO4 (100 mM)
250 μl Bst 3.0 (8 U/ul)
20 ml ddH2O

### Hybridization Chain Reaction

In initial experiments, hybridization chain reaction (HCR), as described in Choi et al. (Development 145:dev165753) was used to detect PONT (POlonies In situ) amplification of the target of interest. HCR has previously been used to identify endogenous nucleotide sequences or sequences amplified by other methods, as described in U.S. Ser. No. 11/087,937. The HCR protocol was performed by incubating the tissue in 100% formamide for 10 minutes at 37° C.; incubating in wash buffer for 15 minutes at room temperature; incubating in hybridization buffer for 15 minutes at 37° C., applying probes in hybridization buffer at a concentration of 0.8 μl/1 00 μl; and incubating overnight at 37° C. The full protocol for three day in situ hybridization chain reaction v3.0 is:

In Situ HCR v3.0—Day 1
1. Incubate in 100% formamide for 10 min @ 37 C
2. Incubate in Wash Buffer for 15 min @ RT
3. Incubate in Hyb Buffer for 15 min @ 37 C
4. Apply Probes in Hyb Buffer (0.8 ul/100 ul)
5. Incubate 0/N @ 37 C

In Situ HCR v3.0—Day 2
1. Prepare and warm the following washing mix to 37 C:

| | |
|---|---|
| 1. | 100% Wash Buffer/0% 5xSSCT |
| 2. | 75% Wash Buffer/25% 5xSSCT |
| 3. | 50% Wash Buffer/50% 5xSSCT |
| 4. | 25% Wash Buffer/75% 5xSSCT |
| 5. | 0% Wash Buffer/100% 5xSSCT |

2. Incubate in Buffer A for 2 min @ 37 C
3. Incubate in Buffer B for 15 min @ 37 C
4. Incubate in Buffer C for 15 min @ 37 C
5. Incubate in Buffer D for 15 min @ 37 C
6. Incubate in Buffer E for 15 min @ 37 C
7. Incubate in 5×SSCT for 5 min @ RT
8. Incubate in Amp Buffer for 30+min @ RT
9. Snap-cool appropriate hairpins
   Aliquot hairpins into PCR tubes
   In a thermal cycler, heat to 95 C for 90 seconds
   Cool to 20 C at a rate of 3 C/min
10. Apply snap-cooled hairpins in Amp Buffer (0.75 ul/100 ul) @RT

In Situ HCR v3.0—Day 3
1. 2× wash w/5×SSCT for 30 min each @RT
2. 1× wash w/5×SSCT for 5 min @RT
3. Seal slides with mounting medium and let dry before imaging

### Example 2: Bridge Amplification and Detection of HPCA mRNA

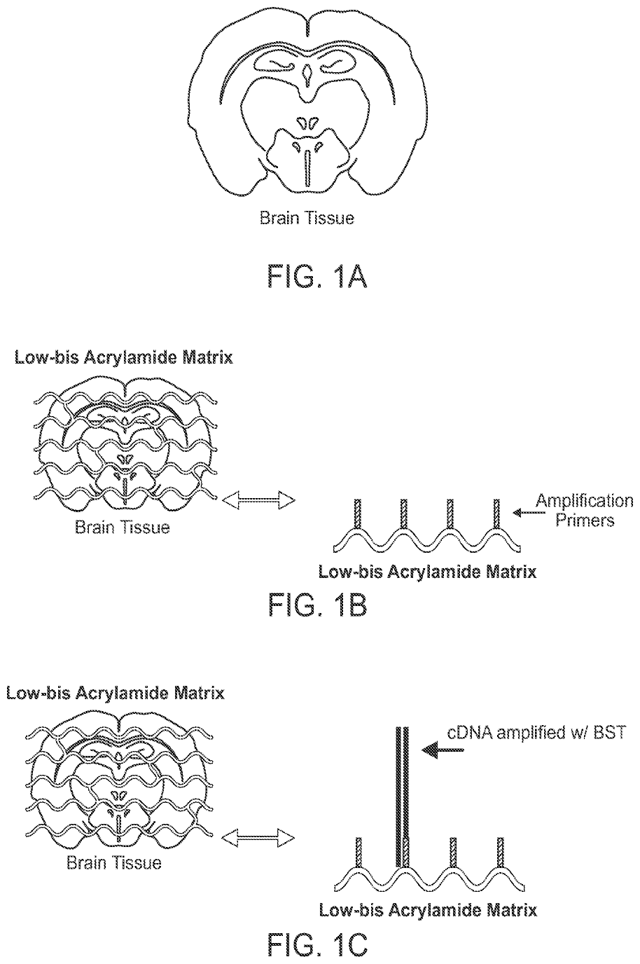

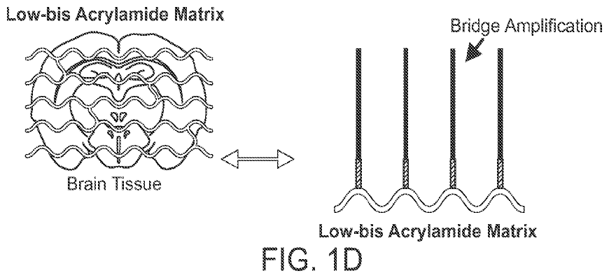

In an initial experiment, HPCA mRNA was detected in situ according to the above Methods described in Example 1. Cryosectioned mouse brain tissue was cut, fixed, and permeabilized. A bis-acrylamide/acrylamide in situ matrix solution (at a ratio of 1:30,000) was prepared containing 5′ acrydite-modified PONI amplification primers targeting HPCA and, this primer-containing matrix solution was added to the prepared fixed and permeabilized tissue (FIGS. 1A-1B). FIG. 1C depicts generation of HPCA cDNAs via binding of matrix-bound primers to endogenous target RNA molecules (here, HPCA RNAs) and reverse transcriptase-mediated primer elongation (optionally including an initial round of amplification). FIG. 1D depicts further amplification of the matrix-associated cDNAs, using bridge amplification (as described in U.S. Ser. No. 12/774,126) to generate HPCA PONIs.

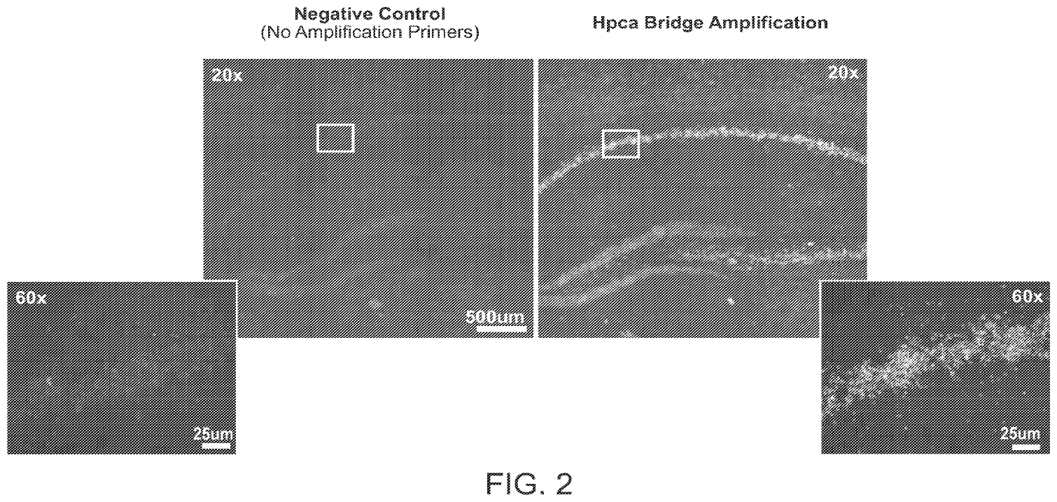

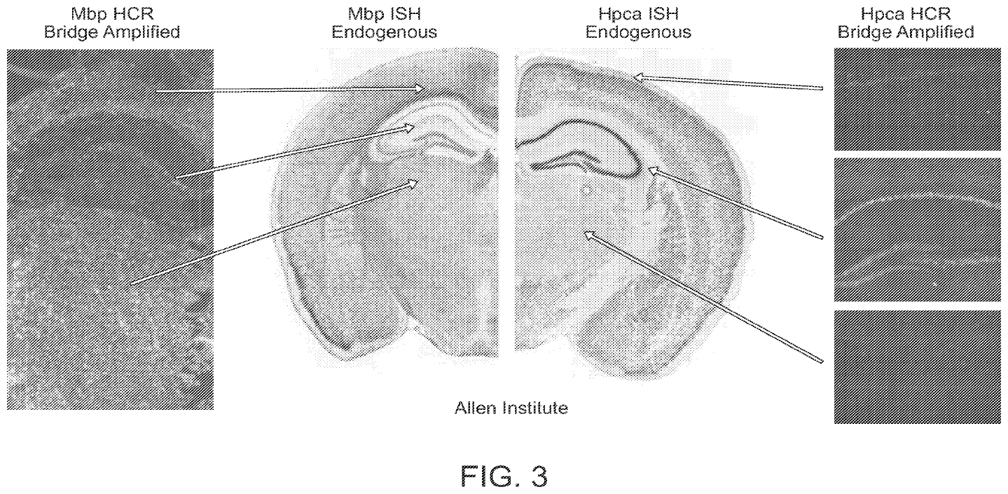

Bridge amplification in situ was then performed, which can be followed by in situ sequencing to detect primer/probe-bound polynucleotides (Example 4 below), and can optionally be followed by detection of the spatial proximity of individually captured molecules (labeled via primer elongation and/or amplification) by promoting and measuring recombination of individual amplicons with nearby amplicons, as recombination between different amplicons will tend to be greater when the amplicons are located in closer proximity to one another, thereby providing spatial information regarding the extent of overlap of detected captured macromolecules (Example 5 below). For the current pilot experiment, the HPCA PONI bridge amplification described herein was detected by in situ DNA-hybridization chain reaction (HCR), with results shown in FIGS. 2-5. In situ bridge amplification maintained spatial specificity of the mRNA transcripts (FIG. 2), as contrasted with a negative control. The distributions of MBP (myelin basic protein) and HPCA mRNAs visualized after in situ bridge amplification using the low-bis in situ matrix solution were compared to reference in situ hybridization (ISH) staining for endogenous Mbp and Hpca transcripts published by the Allen Institute, and the current results were observed to be both consistent with this ISH reference and highly resolved (FIG. 3).

### Example 3: Optimization of In Situ Matrices

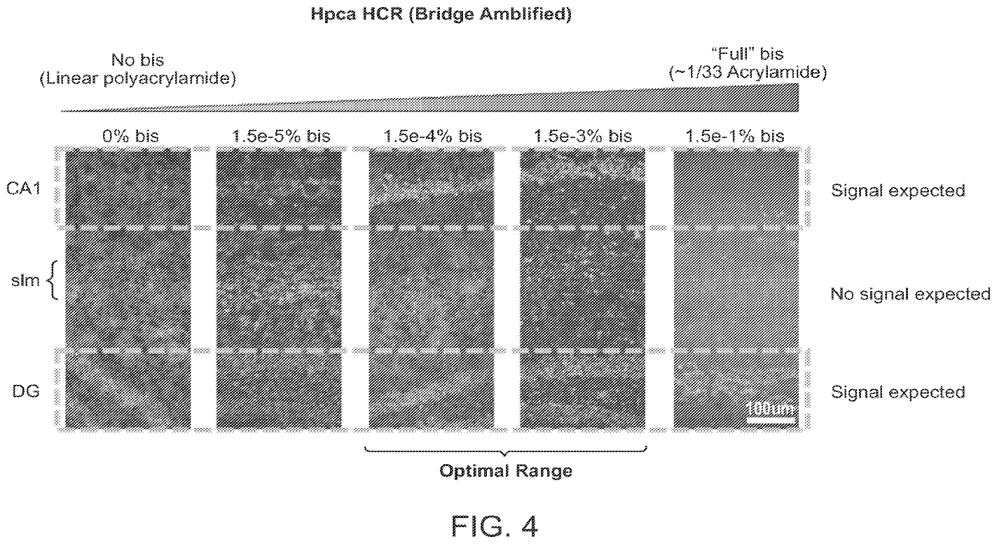

The in situ matrix solution disclosed herein serves to provide structural integrity (as currently exemplified, for bridge amplification and PONI formation and detection with spatial resolution) while maintaining porosity to allow for passage of enzymes into a matrix-contacted tissue. To optimize such in situ matrix solutions for high resolution of tissue features via the current bridge amplification/PONI approach, the ratio of cross-linking agent to linear polymer was varied across a range of bis-acrylamide concentrations, with results of such optimization experiments shown in FIG. 4. HPCA mRNA expression was specifically detected through in situ bridge amplification using primer-containing matrix solutions having percentages of bis-acrylamide to total acrylamide in solution of 0% bis-acrylamide to 0.15% bis-acrylamide. The optimal percentage of bis-acrylamide to total acrylamide in solution in these exemplary experiments was observed to be about 1.5×10⁻⁴% to 1.5×10⁻³% bis-acrylamide to about 5% acrylamide, or about 1:33,000 to about 1:3,300 bis-acrylamide:acrylamide, with about 1:30,000 approximating the matrix solutions that showed the best resolutions obtained (FIG. 4). In such matrix-optimization experiments, matrix-associated HPCA PONIs were labeled with fluorescent probes using in situ DNA-hybridization chain reaction (HCR). It is expressly contemplated that other cross-linking agents can also be used in combination with linear polymers (acrylamide or otherwise), to create similarly porous but spatially defined matrix solutions/matrices. It is further contemplated that useful ratios of various combinations of cross-linking agent to linear polymers or monomers could be as low as about 1:1,000,000 [of cross-linking agent: linear monomer or polymer], or as high as about 1:30 [of cross-linking agent: linear monomer or polymer].

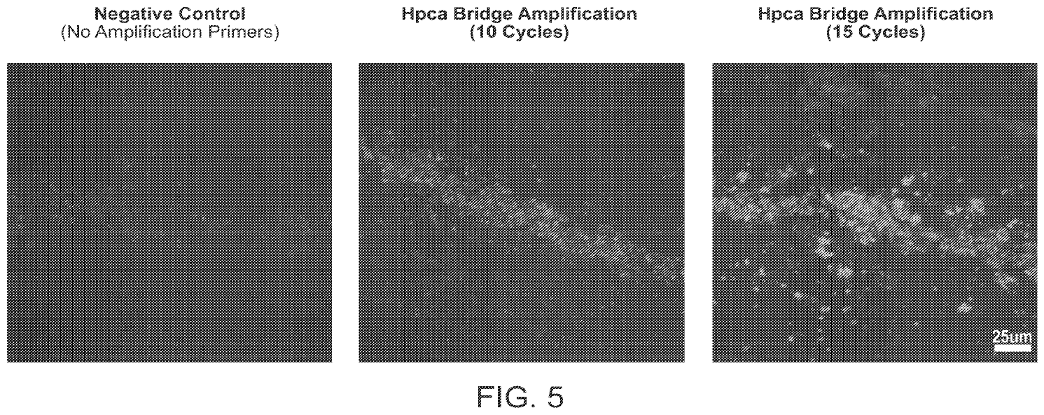

In situ bridge amplification PONI count and size was also observed to depend upon the number of bridge amplification cycles performed, as 10 cycles of bridge amplification produced less intense but smaller (more highly resolved) PONIs, while 15 cycles of bridge amplification produced visibly larger PONIs with higher signal, when detected by HCR (FIG. 5).

### Example 4: In Situ Sequencing-by-Synthesis of PONIs

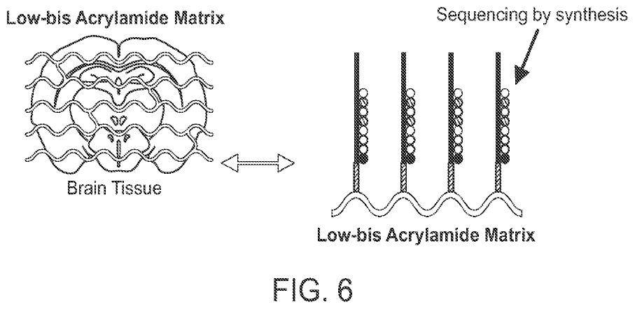

The above Examples demonstrated that individual target mRNAs could be captured and resolved at high spatial resolution using the in situ matrix solution of the instant disclosure with a process of bridge amplification that produced matrix-associated PONIs that could be labeled with fluorescent probes for detection of target molecules. To assess a wide array of target nucleic acids within an individual tissue sample (e.g., tissue section), in situ sequencing can be performed upon a population of matrix-associated PONIs. In particular, in situ sequencing is performed upon matrix-associated PONIs using sequencing-by-synthesis (SBS) (FIG. 6 depicts fluorescent-based SBS), thereby providing almost unlimited parallelization capability, theoretically restricted only by the resolution of the imaging system. In practice, successful implementation of sequencing by synthesis (SBS) is effectively dependent on the read length of the target DNA template. One of the major factors that determines the read length when performing SBS is the number of available templates. Because the magnitude of PONI amplification is controllable in bridge amplification in situ, as described herein, the currently disclosed methods are capable of producing optimal template levels for SBS, thereby facilitating parallelization in detection of captured nucleic acids.

### Example 5: Detection of Spatial Proximity from Amplicon-Amplicon Recombination Rates

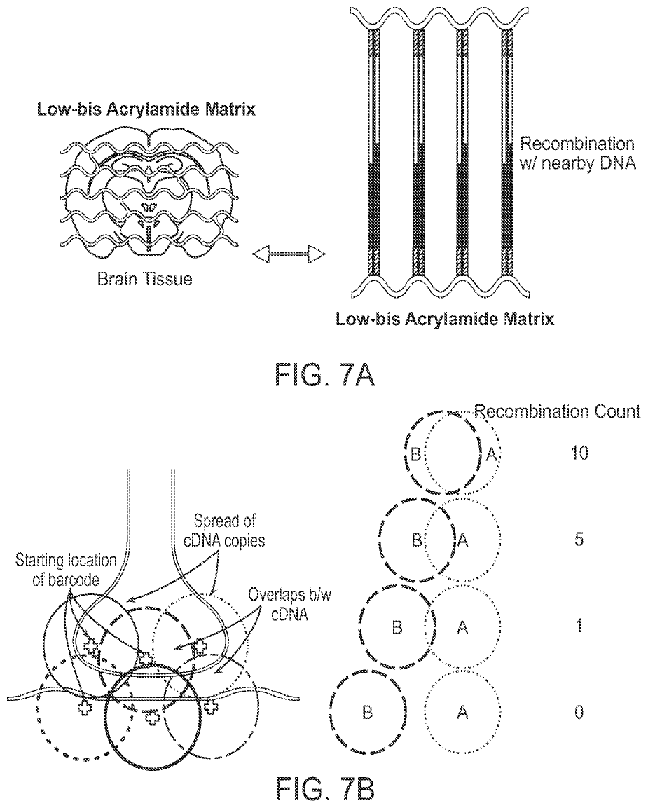

Spatial proximity information for distinct amplicons and/or distinct PONIs can be obtained by detecting amplicon-amplicon recombination rates, during or after initial rounds of bridge amplification are performed. In particular, spatial proximity can be recorded by bridge amplifying the nucleic acids using primers containing overlapping overhangs. With such overlapping overhangs present, nearby amplicons are able to recombine with each other as they further amplify. The closer the two DNA sequences, the more likely they are to be recombined on the same amplicon. FIG. 7A depicts the combining of two nearby DNA sequences during amplification. FIG. 7B depicts the relationship between the spatial proximity of barcode-containing amplicons and the number of recombination events. Thus, newly recombined molecules will then contain sequences of both amplicons. This information can then be read downstream via sequencing to determine which molecules were within a certain spatial distance of each other. Furthermore, the rate of recombination events between individual primary amplicons should decline as a function of increasing spatial distance. Therefore, the number of recombinations (concatamer formations) between two molecules (or amplicons thereof) can serve as a proxy for distance. As described in Weinstein et al. (DNA Microscopy: Optics-free Spatio-genetic Imaging by a Stand-Alone Chemical Reaction. Cell. vol 178(1) 2019), an algorithm has been previously disclosed that decodes molecular proximities from recombined sequences and infers physical images of original transcripts at cellular resolution with precise sequence information. Spatial proximity information may therefore be determined from PONIs using the instant method upon any tissue sample, with an exemplary tissue sample of relevance for the spatial genomics methods disclosed herein being a neuronal tissue sample having individual nerve termini and synapses, where in situ detection of spatial genomic profiles across individual synapses is likely to prove particularly useful.

### Example 6: In Situ Hybridization and Detection of PONIs Robustly Identified Interactions and Proximity Between Biomolecules

To illustrate the ability of the instant compositions and methods to discover interactions or proximity between biomolecules, interactions between proteins and RNA were assessed using the in situ hybridization, amplification and detection methods disclosed herein. For this study, antibodies were conjugated with TotalSeq™ B oligonucleotides (antibody-oligonucleotide conjugated anti-CD200 as a test agent, while an oligonucleotide-conjugated kappa isotype antibody was used as a control), and these antibody-oligonucleotide conjugates were employed to contact mouse brain tissue sections. The CD200 antibody is a surface protein expressed broadly in endothelial cells and some neurons. The control kappa isotype antibody is an immunoglobulin of unknown specificity that has been shown not to label any targets in mouse tissue. It is also contemplated that custom conjugated antibodies using custom oligonucleotides can also be used for the currently disclosed PONI detection methods. Here, tissues were immunolabeled using conjugated antibodies before proceeding with the standard PONI generation and detection protocol. Samples were sequenced using a next-generation sequencing (NGS) system (Illumina™ NextSeg™).

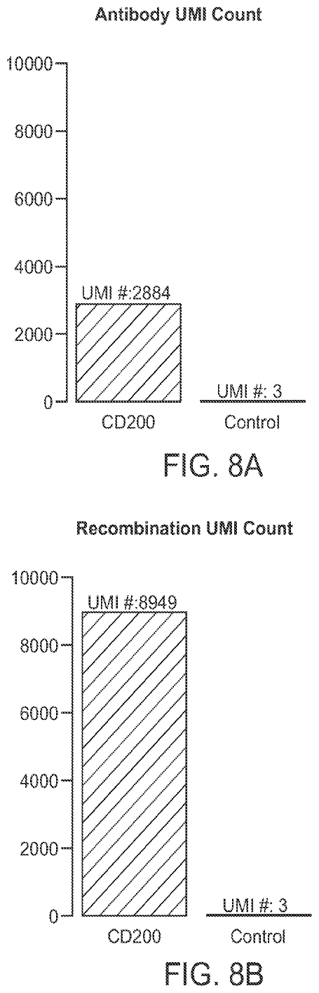

UMI counts respectively identifying anti-CD200 antibody and control antibody were obtained and compared, to assess the signal-to-noise of immunolabeling in PONI-processed tissue (FIG. 8A). Notably, the UMI counts of anti-CD200 antibody were vastly greater than the UMI counts for the control antibody, thereby demonstrating that the anti-CD200 antibody retained specificity after all of the enzymatic processes involved in PONT. UMI counts of recombination events were also identified for each antibody (FIG. 8B). Notably, the UMI count for the anti-CD200 antibody-RNA recombination was greater than the UMI count for the anti-CD200 antibody (˜3× fold). This result demonstrated the ability of an individual molecule to recombine with multiple distinct neighboring molecules during the in situ bridge amplification process.

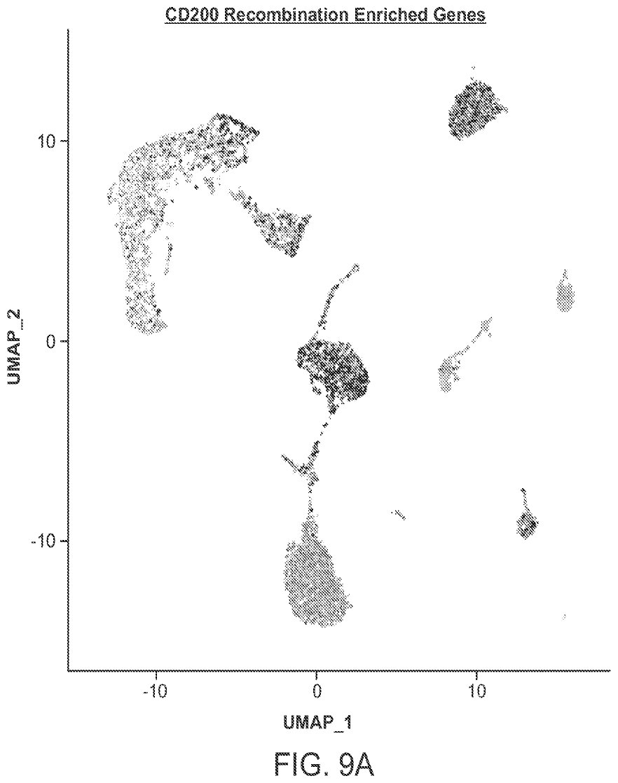

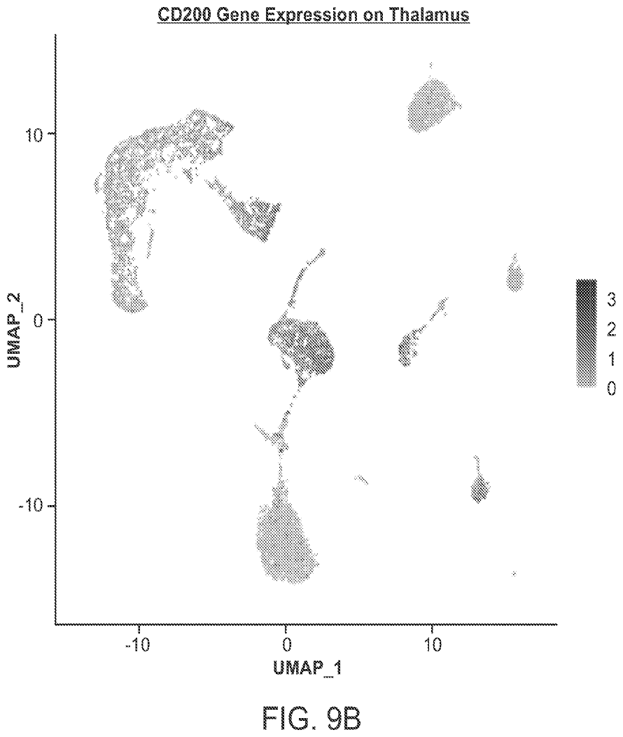

The library of cDNA that recombined with oligonucleotide-conjugated anti-CD200 antibody was compared against the library of total cDNA in the same sample. The top 15 anti-CD200-recombination enriched genes and the top 15 anti-CD200-recombination under-enriched genes were identified. The anti-CD200-recombination enriched and under-enriched gene sets were plotted onto a single-cell dataset of a mouse thalamus (FIG. 9A). Cells were labeled as containing mostly enriched genes (red), under-enriched genes (green), balanced (yellow), or neither (grey). CD200 gene expression was also plotted onto the same single-cell dataset (FIG. 9B). Notably, the anti-CD200-recombination enriched gene set almost completely matched the expression of CD200.

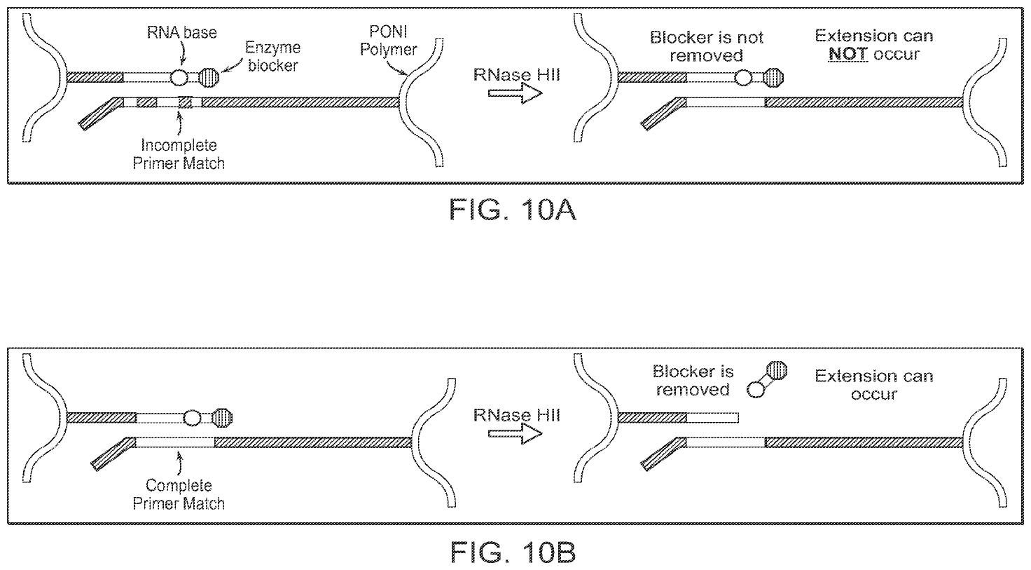

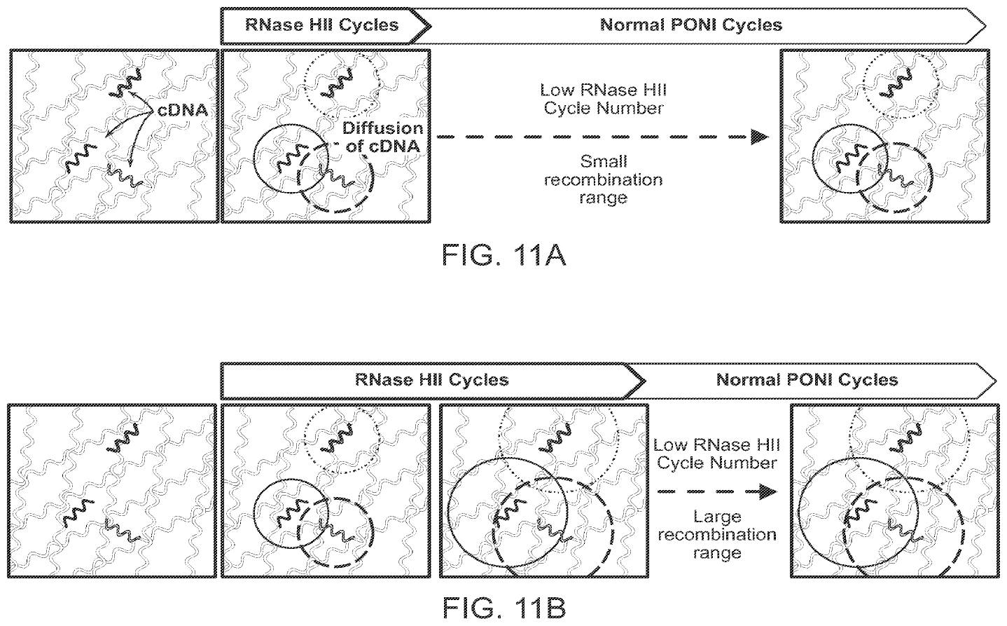

One of the objectives of the current in situ hybridization and PONI detection methods is to identify biomolecular interactions. It is therefore important for enhancing the technology to reduce noise to increase sensitivity and to limit spatial diffusion to increase specificity. Application of RNase HII PCR (rhPCR) as set forth in Dobosy et al. (BMC Biotechnology 11: 80) to the current methods is accordingly contemplated herein to greatly improve both sensitivity and specificity of the currently disclosed PONI assay. In such an approach, PONI primers are designed with an enzymatic blocker on the 3′ end, along with a single RNA base close to the 3′ end. During PONI amplification, the tissue is contacted with the endoribonuclease RNase HIT. If the PONI primer anneals to a template DNA with a complete match, the RNase HII is able to cleave the primer at the single RNA base, thereby removing the enzymatic blocker and allowing extension to proceed in the subsequent polymerase step (FIG. 10B). If the PONI primer anneals to a template DNA with an incomplete match or does not anneal to a DNA, the RNase HII does not act on the PONI primer, thereby retaining the enzymatic blocker and preventing extension from occurring. In this way, the use of rhPCR in PONI amplification ensures that only the intended DNA strands are amplified. Furthermore, because the PONI primers depend on RNase HII to remove the enzymatic blocker in order to amplify, the number of bridge amplification cycles that employ RNase HII can be modulated and used to limit the spatial diffusion of PONT amplicons to fit the user's need. PONT amplification cycles without RNase HII can be subsequently added, to the extent such further cycles are productive to enrich for recombination events without increasing diffusion (FIGS. 11A, 11B).

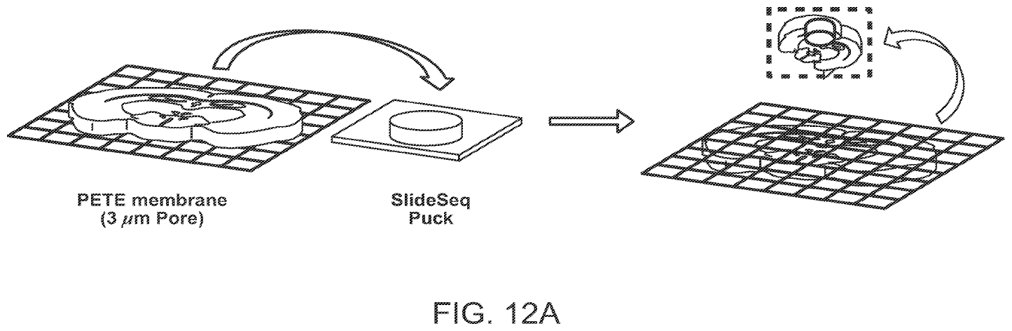

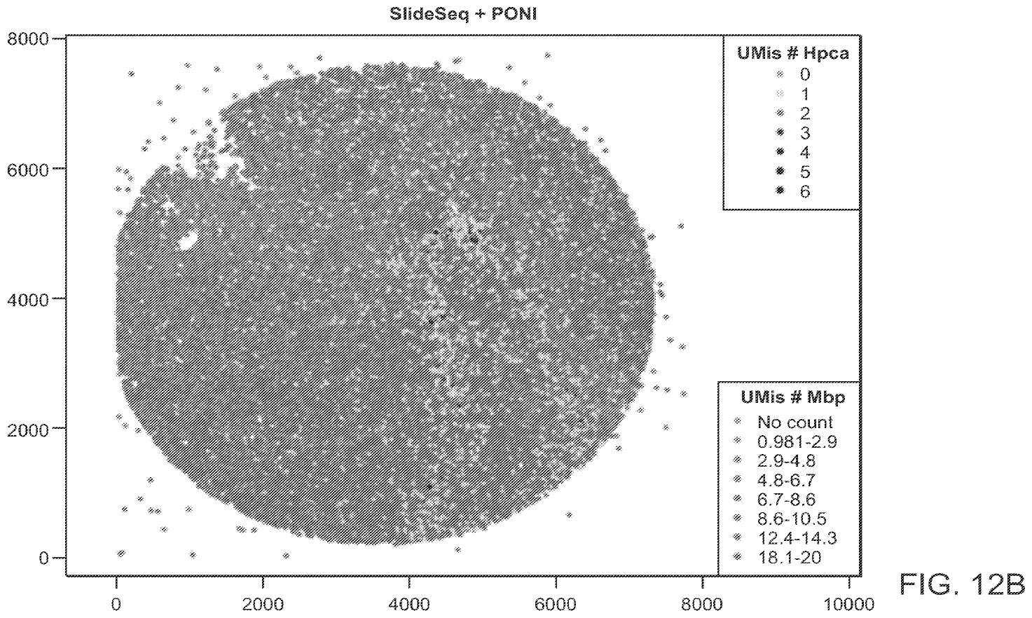

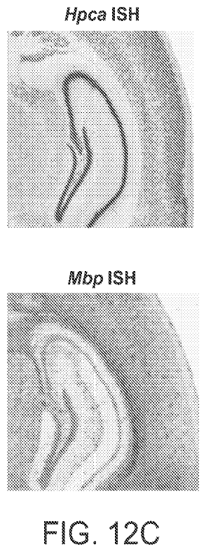

Due to instant PONT generation and detection method's ability to identify biomolecular interactions in situ, the method is contemplated to provide a powerful synergy with in situ spatial transcriptomic methods. To apply in situ spatial transcriptomics to the instant PONT generation and detection approach, the previously disclosed “SlideSeq” method (Stickels et al. Nature Biotechnology 39: 313-319; see also PCT/US19/30194) was adapted for contact with and resolution of PONI-processed tissue (FIG. 12A). For the current SlideSeq experiment, tissue was processed on a permeable and transparent PETE (polyester track etch) membrane. The tissue/membrane was then transferred to a SlideSeq puck for the standard SlideSeq protocol (for spatial transcriptome detection). For the initial study, the whole transcriptome of a mouse brain tissue was PONI-amplified. PONI-amplified Hpca and Mbp UMIs detected by SlideSeq were plotted (FIG. 12B) and compared against in-situ hybridization (ISH) data (FIG. 12C) obtained from the Allen Brain Atlas (available at mouse.brain-map.org). This comparison between the SlideSeq and ISH data revealed a great consistency in the spatial distribution of both Hpca and Mbp, thereby demonstrating the instant PONI approach's compatibility with SlideSeq.

### Example 7: Tissue Section Processing Via a Puck Stack Protocol

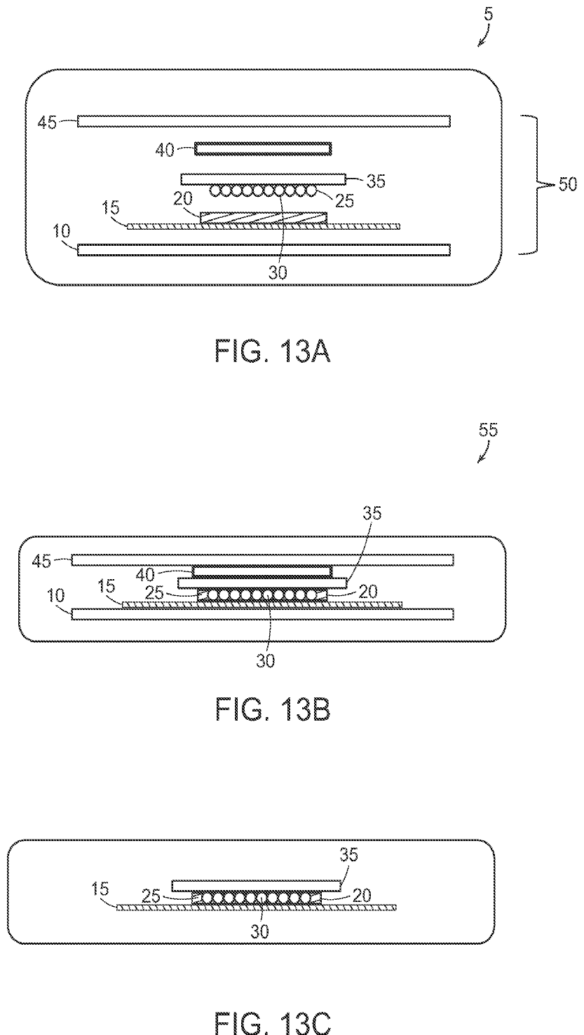

To allow tissue sections to be processed with Slide-seq, a puck stack 5 was formed, as shown in FIG. 13A. Briefly, a section was cut from a frozen tissue 20 and melted onto a porous membrane 15, which may be a permeable PETE (polyester track etch) membrane having pore sizes ranging from 3 μm to 10 μm, however, membrane 15 may be any of a variety of suitable membrane types, including, for example and without limitation, PTFE (polytetrafluoroethylene), PVDF (polyvinylidene fluoride), PES (polyethersulfone), PP (polypropylene) and GF(glass fiber), among others. It is specifically contemplated that membrane compatibility may change depending upon the specific protocol and type of tissue section employed—e.g., for a xylene-treated formalin-fixed paraffin-embedded (FFPE) tissue, PES would no longer be a compatible membrane type. It is contemplated that any such membranes having pore sizes between as small as 0.1 μm and as large as 10 μm, or even larger can be used. In some embodiments, membrane 15 may have pore sizes that are 3 μm, 4 μm, 5 μm, 6 μm, 7 μm, 8 μm, 9 μm, or 10 μm. In some embodiments, membrane 15 may have pore sizes of 3 μm or 10 μm. Once the tissue 20 has melted onto the membrane 15, it may be processed using the PONI protocol disclosed herein (thereby associating the tissue section with a matrix as disclosed herein and associated PONI nucleic acid primers and/or probes) inside a 24-well culture plate or on a slide using hydrophobic gaskets.

When tissue 20 is ready for spatial transcriptomics, tissue 20 and membrane 15 may be placed on top of a first microscope slide 10 with the tissue 20 facing up so that membrane 15 is positioned between tissue 20 and the first slide 10. A drop (˜2 ul) of water is placed on the tissue. A Slide-seq puck 25 including a plurality of beads 30 may be mounted on a coverslip 35, which may then be placed on top of the tissue 20 with the puck 25 facing down towards the tissue 20. In other words, puck 25 may be positioned between tissue 20 and coverslip 35. A spacer element 40 may then optionally be placed on top of puck 25. Spacer element 40 may be a paper spacer having a thickness of between about 0.1 and 0.3 mm. Optionally, a second microscope slide 45 may then be placed on top of paper spacer 40. This initial stack 50 may then be placed inside of a microscope slide press (not shown) and pressed together for about 8 minutes. It is contemplated that the puck stack can be pressed for as little as 1 minute or less, for as long as an hour or more, or for any duration of time in between, e.g., for any amount of time less than about two hours, for any amount of time between 1 minute and 1+ hours, e.g., for 2 minutes to 30 minutes, etc. In addition, it is contemplated that the amount of force/pressure placed upon the puck stack by the slide press can range from about 1N to about 1000N or more and/or from about 0.2 psi to about 220 psi or more, with the upper end of the force/pressure range effectively being the level of force required to crush the beads of the bead array, i.e., any pressure that is insufficient to crush the bead array of the puck stack can be employed. After the initial stack 50 is pressed together, compressed stack 55 is formed, as shown in FIG. 13B. Compressed stack 55 may then be removed from the press, and tissue 20 and Slide-seq puck 25 may then be removed from the compressed stack 55, as shown in FIG. 13C, and processed following the Slide-seq protocol described elsewhere herein and previously (i.e., NGS sequencing is performed upon captured target nucleic acids of the bead array, with spatial identities of the individual beads used to reconstruct spatial information of the bead array-associated target nucleic acids from the bulk-sequenced beads).

Thus, the instant methods provide for accurate and precise in situ DNA amplification and detection of nucleic acid molecules (including endogenous nucleic acids and/or nucleic acid-tagged molecules (e.g., nucleic acid-tagged antibodies that bind specific target proteins within a tissue sample) within any target tissue to which the compositions and methods of the instant disclosure are applied.

The current compositions and methods can be used to understand the nucleic acid profiles of tissues in health and disease, with exemplary applications including: 1) Use for studying how gene expression changes in tissue in response to perturbation and disease. 2) Use for studying how macromolecule distributions in tissue change in response to perturbation and disease. 3) Use for studying developmental, post-mortem, clinical, forensic, and paleoarcheology samples and in particular, use to determine the amplitude, sequence, and localization of nucleic acids in the sample.

Application of the current compositions and methods to detect the abundance, sequence, and/or localization of nucleic acids in tissues, potentially as a diagnostic tool, or as a tool for developing diagnostic assays, or for pathological staging, for diseases (e.g., the instant approach can be used to profile many cancer sections (optionally alongside normal control sections), to reveal a signature of nucleic acid abundance, sequence, and/or localization predictive of disease course and/or treatment response) is also expressly contemplated.

All patents and publications mentioned in the specification are indicative of the levels of skill of those skilled in the art to which the disclosure pertains. All references cited in this disclosure are incorporated by reference to the same extent as if each reference had been incorporated by reference in its entirety individually.

One skilled in the art would readily appreciate that the present disclosure is well adapted to carry out the objects and obtain the ends and advantages mentioned, as well as those inherent therein. The methods and compositions described herein as presently representative of preferred embodiments are exemplary and are not intended as limitations on the scope of the disclosure. Changes therein and other uses will occur to those skilled in the art, which are encompassed within the spirit of the disclosure, are defined by the scope of the claims.

In addition, where features or aspects of the disclosure are described in terms of Markush groups or other grouping of alternatives, those skilled in the art will recognize that the disclosure is also thereby described in terms of any individual member or subgroup of members of the Markush group or other group.

The use of the terms “a” and “an” and “the” and similar referents in the context of describing the disclosure (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein.

All methods described herein can be performed in any suitable order unless otherwise indicated herein or otherwise clearly contradicted by context. The use of any and all examples, or exemplary language (e.g., “such as”) provided herein, is intended merely to better illuminate the disclosure and does not pose a limitation on the scope of the disclosure unless otherwise claimed. No language in the specification should be construed as indicating any non-claimed element as essential to the practice of the disclosure.

Embodiments of this disclosure are described herein, including the best mode known to the inventors for carrying out the disclosed invention. Variations of those embodiments may become apparent to those of ordinary skill in the art upon reading the foregoing description.

The disclosure illustratively described herein suitably can be practiced in the absence of any element or elements, limitation or limitations that are not specifically disclosed herein. Thus, for example, in each instance herein any of the terms “comprising”, “consisting essentially of”, and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present disclosure provides preferred embodiments, optional features, modification and variation of the concepts herein disclosed may be resorted to by those skilled in the art, and that such modifications and variations are considered to be within the scope of this disclosure as defined by the description and the appended claims.

It will be readily apparent to one skilled in the art that varying substitutions and modifications can be made to the invention disclosed herein without departing from the scope and spirit of the invention. Thus, such additional embodiments are within the scope of the present disclosure and the following claims. The present disclosure teaches one skilled in the art to test various combinations and/or substitutions of chemical modifications described herein toward generating conjugates possessing improved contrast, diagnostic and/or imaging activity. Therefore, the specific embodiments described herein are not limiting and one skilled in the art can readily appreciate that specific combinations of the modifications described herein can be tested without undue experimentation toward identifying conjugates possessing improved contrast, diagnostic and/or imaging activity.

The inventors expect skilled artisans to employ such variations as appropriate, and the inventors intend for the disclosure to be practiced otherwise than as specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context. Those skilled in the art will recognize, or be able to ascertain using no more than routine experimentation, many equivalents to the specific embodiments of the disclosure described herein. Such equivalents are intended to be encompassed by the following claims.

1. A composition comprising:
(i) a first monomer or linear polymer;
(ii) a cross-linking agent comprising a second monomer or polymer, wherein the cross-linking agent is capable of crosslinking with the first monomer or linear polymer when combined; and
(iii) a nucleic acid primer or probe comprising a modification capable of binding or chemically conjugating the primer or probe to the first monomer or linear polymer, the cross-linking agent, or both,
wherein the ratio of the cross-linking agent to the first monomer or linear polymer is between about 1:1,000,000 and about 1:30 by weight.

2. The composition of claim 1, wherein the first monomer or linear polymer comprises one or more compounds selected from the group consisting of acrylamide, methacrylate, polyethylene glycol (PEG), carboxymethyl cellulose (CMC), polyvinylpyrrolidone (PVP), isopropylacrylamide, hyaluronic acid, heparin, polylactic acid (PLA), polyglycolide (PGA), and poly(lactic-co-glycolic acid) (PLGA), Polyhydroxyalkanoates (PHA), propylene fumarate (PPF), agarose, alginate, chitosan, ethylene glycol-decorated polyisocyanide (PIC) polymers, derivatives thereof, and combinations thereof.

3. The composition of claim 1, wherein the cross-linking agent comprises one or more compounds selected from the group consisting of N,N′-methylene bisacrylamide, trisacrylamide, tetracrylamide, polyethylene glycol dimethacrylate, amine end-functionalized 4-arm star-PEG, derivatives thereof, and combinations thereof.

4. The composition of claim 3, wherein the polyethylene glycol dimethacrylate comprises triethylene glycol dimethyacrylate (TEGDMA), tetra(ethylene glycol) dimethacrylate, or both.

5. The composition of claim 1, wherein the ratio of the cross-linking agent to the first monomer or linear polymer is at most 1:30 by weight, optionally wherein the ratio of the cross-linking agent to the first monomer or linear polymer is at most 1:50 by weight, optionally wherein the ratio of the cross-linking agent to the first monomer or linear polymer is at most 1:100 by weight, optionally wherein the ratio of the cross-linking agent to the first monomer or linear polymer is at most 1:200 by weight, optionally wherein the ratio of the cross-linking agent to the first monomer or linear polymer is at most 1:500 by weight, optionally wherein the ratio of the cross-linking agent to the first monomer or linear polymer is at most 1:1000 by weight, optionally wherein the ratio of the cross-linking agent to the first monomer or linear polymer is at most 1:2,000 by weight, optionally wherein the ratio of the cross-linking agent to the first monomer or linear polymer is at most 1:3,000 by weight, optionally wherein the ratio of the cross-linking agent to the first monomer or linear polymer is at most 1:5,000 by weight, optionally wherein the ratio of the cross-linking agent to the first monomer or linear polymer is at most 1:10,000 by weight, optionally wherein the ratio of the cross-linking agent to the first monomer or linear polymer is at most 1:30,000 by weight, optionally wherein the ratio of the cross-linking agent to the first monomer or linear polymer is at most 1:50,000 by weight, optionally wherein the ratio of the cross-linking agent to the first monomer or linear polymer is at most 1:100,000 by weight, optionally wherein the ratio of the cross-linking agent to the first monomer or linear polymer is at most 1:300,000 by weight, optionally wherein the ratio of the cross-linking agent to the first monomer or linear polymer is at most 1:500,000 by weight, optionally wherein the ratio of the cross-linking agent to the first monomer or linear polymer is at most 1:750,000 by weight, optionally wherein the ratio of the cross-linking reagent to the to the first monomer or linear polymer is at most 1:1,000,000 by weight.

6. The composition of claim 1, wherein the modification is a phosphoramidite modification, optionally an acrydite modification.

7. The composition of claim 1, wherein the nucleic acid primer or probe binds or chemically conjugates to the first monomer or linear polymer, optionally wherein the nucleic acid primer or probe covalently binds or chemically conjugates to the first monomer or linear polymer, optionally wherein the first monomer or linear polymer is acrylamide.

8. The composition of claim 1, further comprising a cell or tissue, optionally wherein the cell or tissue is a fixed and/or permeabilized cell or tissue.

9. The composition of claim 8, wherein the cell or tissue is a tissue section, optionally wherein the tissue section is a cryosection or a fixed tissue section, optionally wherein the fixed tissue section is a formalin-fixed tissue section, optionally wherein the formalin-fixed tissue section is a formalin-fixed paraffin-embedded (FFPE) tissue section, optionally wherein the FFPE tissue section has been treated with xylene to remove paraffin.

10. The composition of claim 1, wherein the nucleic acid primer or probe comprises a barcode sequence and/or a unique molecular identifier (UMI) sequence.

11. The composition of claim 1, wherein the nucleic acid primer or probe comprises a poly-T sequence.

12. The composition of claim 1, wherein:
the nucleic acid primer or probe comprises a 3′-terminus possessing an enzymatic blocker and at least one RNA base in sufficiently close proximity to the 3′-terminus for a RNase HII enzyme to remove both the enzymatic blocker and the at least one RNA base if the nucleic acid primer or probe specifically anneals with a target nucleic acid molecule, thereby forming a double-stranded substrate for the RNase HII enzyme;
the first monomer or linear polymer is acrylamide,
the cross-linking agent comprising a second monomer or polymer is N,N′-methylene bisacrylamide, optionally wherein the ratio of N,N′-methylene bisacrylamide to acrylamide is about 1:50,000 to about 1:30, optionally wherein the ratio of N,N′-methylene bisacrylamide to acrylamide is about 1:40,000 to about 1:100, optionally wherein the ratio of N,N′-methylene bisacrylamide to acrylamide is about 1:35,000 to about 1:500, optionally wherein the ratio of N,N′-methylene bisacrylamide to acrylamide is about 1:30,000 to about 1:1,000, optionally wherein the ratio of N,N′-methylene bisacrylamide to acrylamide is about 1:25,000 to about 1:2,500, optionally wherein the ratio of N,N′-methylene bisacrylamide to acrylamide is about 1:20,000 to about 1:5,000, optionally wherein the ratio of N,N′-methylene bisacrylamide to acrylamide is about 1:16.667.

13. The composition of claim 1, further comprising:
reverse transcriptase, a DNA polymerase and/or a RNase HII enzyme; and/or
tetramethylethylenediamine (TEMED), optionally further comprising ammonium persulfate (APS) or riboflavin.

14-18. (canceled)

19. A method for binding a target nucleic acid molecule of or associated with a tissue, the method comprising:
(i) providing a tissue;
(ii) contacting the tissue with a first monomer or linear polymer; a cross-linking agent comprising a second monomer or polymer, wherein the cross-linking agent is capable of crosslinking with the first monomer or linear polymer when combined; and a nucleic acid primer or probe comprising a modification capable of binding the primer or probe to the first monomer or linear polymer, the cross-linking agent, or both, wherein the ratio of the cross-linking agent to the first monomer or linear polymer is between about 1:1,000,000 and about 1:30 by weight;
(iii) crosslinking the cross-linking agent with the first monomer or linear polymer, thereby forming a matrix;
(iv) binding the nucleic acid primer or probe to the first monomer or linear polymer, the cross-linking agent, or both;
(v) incubating the matrix and nucleic acid primer or probe with the tissue under conditions suitable for annealing of the nucleic acid primer or probe to a target nucleic acid molecule of or associated with the tissue, thereby forming a primer-bound or probe-bound target nucleic acid molecule,
thereby binding a target nucleic acid molecule of or associated with the tissue.

20. The method of claim 19, wherein:
the tissue is a tissue section, optionally wherein the tissue section is a cryosection or a fixed tissue section, optionally wherein the fixed tissue section is a formalin-fixed tissue section, optionally wherein the formalin-fixed tissue section is a formalin-fixed paraffin-embedded (FFPE) tissue section, optionally wherein the FFPE tissue section has been treated with xylene to remove paraffin;
the nucleic acid primer or probe comprises a barcode sequence and/or a unique molecular identifier (UMI) sequence;
the nucleic acid primer or probe comprises a poly-T sequence;
the nucleic acid primer or probe comprises a 3′-terminus possessing an enzymatic blocker and at least one RNA base in sufficiently close proximity to the 3′-terminus for a RNase HII enzyme to remove both the enzymatic blocker and the at least one RNA base if the nucleic acid primer or probe specifically anneals with a target nucleic acid molecule, thereby forming a double-stranded substrate for the RNase HII enzyme;
the method further comprises (vi) contacting the primer-bound or probe-bound target nucleic acid molecule with one or more enzymes selected from the group consisting of reverse transcriptase, a DNA polymerase, and RNase HIT;
the first monomer or linear polymer is acrylamide, optionally wherein the cross-linking agent comprising a second monomer or polymer is N,N′-methylene bisacrylamide, optionally wherein the ratio of N,N′-methylene bisacrylamide to acrylamide is about 1:50,000 to about 1:30, optionally wherein the ratio of N,N′-methylene bisacrylamide to acrylamide is about 1:40,000 to about 1:100, optionally wherein the ratio of N,N′-methylene bisacrylamide to acrylamide is about 1:35,000 to about 1:500, optionally wherein the ratio of N,N′-methylene bisacrylamide to acrylamide is about 1:30,000 to about 1:1,000, optionally wherein the ratio of N,N′-methylene bisacrylamide to acrylamide is about 1:25,000 to about 1:2,500, optionally wherein the ratio of N,N′-methylene bisacrylamide to acrylamide is about 1:20,000 to about 1:5,000, optionally wherein the ratio of N,N′-methylene bisacrylamide to acrylamide is about 1:16,667;
step (iii) comprises contacting the cross-linking agent and the first monomer or linear polymer with tetramethylethylenediamine (TEMED), optionally wherein the method further comprises contacting the cross-linking agent and the first monomer or linear polymer with ammonium persulfate (APS) or riboflavin;
the nucleic acid primer or probe is incubated with the tissue under conditions suitable for amplification of the primer-bound or probe-bound target nucleic acid molecule;
the primer-bound or probe-bound target nucleic acid is bridge amplified, optionally wherein bridge amplification is performed in a flowcell;
a population of distinct individual target molecules is amplified;
the target molecule is a mRNA,
the target molecule is a nucleic acid-tagged polypeptide, optionally a nucleic acid-tagged antibody;
the target nucleic acid is amplified for between five and fifty amplification cycles, optionally wherein the target nucleic acid is amplified for between five and twenty amplification cycles, optionally wherein the target nucleic acid is amplified for between ten and fifteen amplification cycles, optionally wherein the amplification cycles are bridge amplification cycles;
RNase HII is added to one or more amplification cycles, optionally wherein RNase HII treatment is performed in a single cycle of bridge amplification, alternatively wherein RNase HII treatment is performed in 2, 3, 4 or more cycles of bridge amplification treatment, optionally wherein the number of bridge amplification cycles that include RNase HII treatment is adjusted by the user to optimize spatial diffusion for a given tissue and collection of target sequences, optionally wherein additional bridge amplification cycles are performed in the absence of RNase HII;
the method further comprises contacting the target nucleic acid or an amplicon of the target nucleic acid with a labeled probe, optionally wherein the labeled probe is a fluorescently labeled probe;
the target nucleic acid or an amplicon of the target nucleic acid is detected, optionally wherein target nucleic acid amplicons are detected with spatial resolution, optionally wherein target nucleic acid amplicons are detected with spatial resolution of about 10 μm or less, optionally about 1 μm or less, optionally about 250 nm or less;
the method further comprises sequencing the target nucleic acid or an amplicon of the target nucleic acid in situ, optionally wherein the sequencing is sequencing-by-synthesis (SBS);
the method further comprises detecting the spatial proximity of target nucleic acids by measuring the frequency of recombination events during bridge amplification between amplicons of different target nucleic acids;
the tissue comprises neuronal synapses;
the method further comprises determining spatial proximity of two or more target nucleic acids by measuring the frequency of recombination events between amplicons of the two or more target nucleic acids during performance of bridge amplification, optionally wherein spatial proximity of the two or more target nucleic acids is detected at a neuronal synapse;
the first monomer or linear polymer comprises one or more compounds selected from the group consisting of acrylamide, methacrylate, polyethylene glycol (PEG), carboxymethyl cellulose (CMC), polyvinylpyrrolidone (PVP), isopropylacrylamide, hyaluronic acid, heparin, polylactic acid (PLA), polyglycolide (PGA), and poly(lactic-co-glycolic acid) (PLGA), Polyhydroxyalkanoates (PHA), propylene fumarate (PPF), agarose, alginate, chitosan, ethylene glycol-decorated polyisocyanide (PIC) polymers, derivatives thereof, and combinations thereof;
the cross-linking agent comprises one or more compounds selected from the group consisting of N,N′-methylene bisacrylamide, trisacrylamide, tetracrylamide, polyethylene glycol dimethacrylate, amine end-functionalized 4-arm star-PEG, derivatives thereof, and combinations thereof, optionally wherein the polyethylene glycol dimethacrylate comprises triethylene glycol dimethyacrylate (TEGDMA), tetra(ethylene glycol) dimethacrylate, or both;
the tissue is fixed with 4% paraformaldehyde (PFA) and/or the tissue is permeabilized with 0.25% Triton;
the method further comprises bridge amplification of the target nucleic acid in a flowcell at 37° C., optionally wherein each cycle of bridge amplification comprises a formamide incubation step and a reverse transcriptase polymerization step, optionally wherein the bridge amplification is performed for between five and fifty cycles;
the method further comprises contacting bridge-amplified target nucleic acids with primers and reversible 3′ fluorescent nucleotide blockers and performing sequencing-by-synthesis;
the method further comprises contacting the matrix with a slide-attached bead array and performing next-generation sequencing (NGS) upon captured target nucleic acids, optionally associating spatial information of the bead array and nucleic acid sequence identities to form an image having spatial resolution of about 50 μm or less, optionally of about 10 μm or less, optionally of about 1 μm or less, optionally of about 250 nm or less; and/or
the method further comprises forming a puck stack comprising a first slide; a membrane; the tissue associated with the matrix; and a puck comprising a bead array attached to a coverslip, wherein the membrane, tissue section associated with the matrix, and puck comprising the bead array attached to the coverslip are sandwiched between the first slide and the coverslip, and the tissue section associated with the matrix is sandwiched between the membrane and the puck comprising the bead array attached to the coverslip, optionally wherein:
the puck stack further comprises a spacer element, optionally wherein the puck comprising the bead array attached to the coverslip, the tissue section associated with the matrix and the membrane are sandwiched between the spacer element and the first slide, optionally wherein the spacer element is a paper spacer, optionally wherein the paper spacer has a thickness of between about 0.1 and 0.3 mm;
the puck stack further comprises a second slide, optionally wherein the puck comprising the bead array attached to the coverslip, the tissue section associated with the matrix and the membrane are sandwiched between the second slide and the first slide, optionally wherein the spacer element is positioned between the second slide and the coverslip and wherein the spacer element, the puck comprising the bead array attached to the coverslip, the tissue section associated with the matrix and the membrane are sandwiched between the second slide and the first slide; and/or
the method further comprises performing next-generation sequencing (NGS) upon captured target nucleic acids of the bead array, optionally associating spatial information of the bead array and nucleic acid sequence identities of target nucleic acids to form an image having spatial resolution of about 50 μm or less, optionally of about 10 μm or less, optionally of about 1 μm or less, optionally of about 250 nm or less.

21-53. (canceled)

54. A kit comprising the composition of claim 1, and instructions for its use.

55. A puck stack, comprising:
a first slide;
a membrane;
a tissue section; and
a puck comprising a bead array attached to a coverslip, wherein the membrane, tissue section, and puck comprising the bead array are sandwiched between the first slide and the coverslip, and the tissue section is sandwiched between the membrane and the puck.

56. The puck stack of claim 55, wherein:
the puck stack further comprises a spacer element, optionally wherein the puck comprising the bead array attached to the coverslip, the tissue section and the membrane are sandwiched between the spacer element and the first slide, optionally wherein the spacer element is a paper spacer, optionally wherein the paper spacer has a thickness of between about 0.1 and 0.3 mm;
the puck stack further comprises a second slide, optionally wherein the puck comprising the bead array attached to the coverslip, the tissue section and the membrane are sandwiched between the second slide and the first slide, optionally wherein the spacer element is positioned between the second slide and the coverslip and wherein the spacer element, the puck comprising the bead array attached to the coverslip, the tissue section and the membrane are sandwiched between the second slide and the first slide; and/or
the tissue section has been processed by a method comprising (i) providing a tissue; (ii) contacting the tissue with a first monomer or linear polymer; a cross-linking agent comprising a second monomer or polymer, wherein the cross-linking agent is capable of crosslinking with the first monomer or linear polymer when combined; and a nucleic acid primer or probe comprising a modification capable of binding the primer or probe to the first monomer or linear polymer, the cross-linking agent, or both, wherein the ratio of the cross-linking agent to the first monomer or linear polymer is between about 1:1,000,000 and about 1:30 by weight (iii) crosslinking the cross-linking agent with the first monomer or linear polymer, thereby forming a matrix; (iv) binding the nucleic acid primer or probe to the first monomer or linear polymer, the cross-linking agent, or both; (v) incubating the matrix and nucleic acid primer or probe with the tissue under conditions suitable for annealing of the nucleic acid primer or probe to a target nucleic acid molecule of or associated with the tissue, thereby forming a primer-bound or probe-bound target nucleic acid molecule and/or matrix associated with the tissue section, optionally wherein the primer-bound or probe-bound target nucleic acid molecule associated with the tissue section has been amplified.

57-58. (canceled)

59. A method of processing a puck stack, comprising:
inserting the puck stack of claim 55 into a slide press;
applying pressure for a period of time; and
creating a compressed puck stack.

60. The method of claim 59, further comprising removing the puck comprising the bead array attached to the coverslip from the compressed puck stack, optionally further comprising performing next-generation sequencing (NGS) upon captured target nucleic acids of the bead array, optionally associating spatial information of the bead array and nucleic acid sequence identities of the target nucleic acids to form an image having spatial resolution of about 50 μm or less, optionally of about 10 μm or less, optionally of about 1 μm or less, optionally of about 250 nm or less.

61. (canceled)
